(19) United States
(12) Patent Application Publication
(10) Pub. No.: US 2016/0279202 A1
(43) Pub. Date: Sep. 29, 2016

(54) INTERLEUKIN-1 (IL-1) INHIBITORS FOR TREATING FERTILITY DISORDERS

(71) Applicants: RAMOT AT TEL-AVIV UNIVERSITY LTD., Tel Aviv (IL); TEL HASHOMER MEDICAL RESEARCH INFRASTRUCTURE AND SERVICES LTD., Ramat Gan (IL)

(72) Inventors: Yehuda KAMARI, Tel Aviv (IL); Ruth SHALGI, Herzliya (IL); Shiri URI, Hod Hasharon (IL); Dror HARATS, Ramat Gan (IL); Aviv SHAISH, Talmei Yehyel (IL)

(73) Assignees: RAMOT AT TEL-AVIV UNIVERSITY LTD., Tel Aviv (IL); TEL HASHOMER MEDICAL RESEARCH INFRASTRUCTURE AND S ERVICES LTD., TEL HASHOMER (IL)

(21) Appl. No.: 15/032,835

(22) PCT Filed: Oct. 30, 2014
(86) PCT No.: PCT/IL2014/050942

§ 371 Date: Apr. 28, 2016

Related U.S. Application Data
(60) Provisional application No. 61/897,990, filed on Oct. 31, 2013.

(51) Int. Cl. A61K 38/19; C12N 5/075

(52) U.S. Cl. CPC A61K 38/19; C12N 5/0609; C12N 2501/2301

(57) ABSTRACT

The invention provides IL-1 inhibitors, methods and uses thereof for retaining ovarian follicle reserve in a mammalian subject, treating infertility and improving responsiveness and sensitivity to COH. The invention further provides methods for improving oocyte quality and/or survival, specifically for IVF, and kits for combining COH treatment with the sensitizing IL-inhibitors of the invention.

### FIELD OF THE INVENTION

The present invention relates to fertility and related conditions. More particularly, the invention provides methods and uses of agents for retaining ovarian follicle reserve, improving response to controlled ovarian hyper stimulation and for treating infertility and related conditions.

### BACKGROUND REFERENCES

1. George J W, Dille E a, Heckert L L (2011) Current concepts of follicle-stimulating hormone receptor gene regulation. Biol Reprod 84:7-17.
2. Gerdes J, Schwab U, Lemke H, Stein H (1983) Production of a mouse monoclonal antibody reactive with a human nuclear antigen associated with cell proliferation. Int J Cancer 31:13-20.
3. Scholzen T, Gerdes J (2000) The Ki-67 Protein: From the Known and. 322:311-322.
4. Bar-Joseph H et al. (2010) Doxorubicin-induced apoptosis in germinal vesicle (GV) oocytes. Reprod Toxicol 30:566-72.
5. Morita et al. (2000) Oocyte apoptosis is suppressed by disruption of the acid sphingomyelinase gene or by sphingosine-1-phosphate therapy. Nat Med 6:1109-14.
6. Bar-Joseph H et al. (2010) Doxorubicin-induced apoptosis in germinal vesicle (GV) oocytes. Reprod Toxicol 30:566-72.
7. Eliyahu E, Park J-H, Shtraizent N, He X, Schuchman E H (2007) Acid ceramidase is a novel factor required for early embryo survival. FASEB J 21:1403-9.

### BACKGROUND OF THE INVENTION

Women, to a greater degree than men are subjected to a biological clock that dictates the end of the reproductive lifespan. On average this occurs at around 50 years of age. Fecundity, i.e., the ability to reproduce, and more specifically, the probability of becoming pregnant in a single menstrual cycle, sharply decreases at age 30. Fecundity may be affected by genetic or environmental factors, or likely both.

There is an upward trend in the number of couples and single women availing themselves of assisted reproductive technologies (ART), for example, in vitro fertilization (IVF) treatments. Many of the IVF treatments may be the result of women postponing their childbearing years. For example, the number of IVF treatment cycles in Israel in 2011 was 38,984. In the years 2009-2011, 4.1% of all live births in Israel were the result of IVF treatment.

However, there are still patients who respond poorly to ART, IVF and more specifically, controlled ovarian hyperstimulation (COH). This poor response may result in only few oocytes at egg retrieval for IVF, a reduced number of embryos available for transfer back into the patient for implantation and a poor resultant pregnancy rate for women undergoing IVF.

Reports of the prevalence of poor responders to COH vary widely between 5.6 and 35.1% of women attempting IVF treatment. In most patients, the underlying cause of this poor response remains unknown.

In general, female mammals, e.g., humans, are born with a finite number of oocytes which gradually decreases during pre-pubertal development and adult life. Inflammation, among other pathologies, has been reported to adversely affect hormone production, ovulation and consequently fertility. Specifically, inflammation has an adverse effect both on IVF outcomes and the ovarian reserve.

Biologically, each oocyte is encircled by somatic granulosa cells (GCs) to form the basic functioning unit of the ovary, i.e., the follicle. An intact nuclear membrane termed ‘germinal vesicle’ (GV) surrounds the genetic material of the oocyte which is arrested at the prophase of the first meiotic division. Ovarian follicles are subdivided into four main categories according to their size and developmental stage; primordial follicle (PMF), primary, secondary and antral follicles. PMFs are in a state of growth arrest and are referred to as the ovarian reserve. All other follicles are known as ‘growing follicles’.

The ovarian functional lifespan is determined by the size of the oocytes “stockpile” provided at birth, as well as by the rate at which this endowment is depleted. Programmed cell death (apoptosis) has been identified as a central mechanism responsible for the age-related exhaustion of oocytes, while a delicate balance between pro-survival and pro-apoptotic molecules determines the final destiny of the follicle. Throughout the entire fertility lifespan in females, starting from fetal life until menopause, over ninety nine percent of the oocytes undergo apoptosis.

Sphingolipid ceramide has emerged as an essential second messenger in the cascade that promotes age-related apoptosis. Lower ceramide levels, observed in acid sphingomyelinase-deficient mice, resulted in a larger pool of oocytes compared to their WT counterparts. Furthermore, studies using Bax null female mice provided evidence that ovarian lifespan can be extended. Despite these reports, the molecular pathways that regulate follicular apoptosis and the consequent reproductive aging remain poorly understood.

The IL-1 family members are among the most potent molecules of the innate immune system. The two major prototypic agonist cytokines in this family, IL-1α and IL-1β, induce the expression of a variety of pro-inflammatory genes upon binding to IL-1 receptor type 1 (IL-1R1). Both IL-1α and IL-1β are synthesized as precursors (31 kDa) and are processed to mature forms (17 kDa) via specific cellular proteases. IL-1α and IL-1β are produced by tissue macrophages, monocytes, fibroblasts, and dendritic cells, B lymphocytes, NK cells and epithelial cells.

IL-1β is generated upon inflammatory signals and is only active as a mature secreted protein after cleavage by caspase-1. In contrast, IL-1α exerts its effects both in the mature and precursor forms when binding to IL-1R1. It is constitutively expressed in the cytosol of epithelial cells, keratinocytes, and fibroblasts, and it is up-regulated during inflammation. IL-1α is a unique dual-function cytokine that in addition to its extracellular receptor-mediated effects, can also translocate to the nucleus, bind to chromatin and possibly affect transcription. IL-1α is also involved in inflammatory responses in cases of ischemia or other instances of sterile inflammation in the absence of infection.

The naturally occurring IL-1 receptor antagonist (IL-1Ra) competes with IL-1α and IL-1β for binding to IL-1RI and thereby down regulates the downstream signaling pathways of IL-1α and IL-1β. Inhibiting IL-1 activity including IL-1RI blocking and/or inhibition is used for treating a number of medical conditions such as rheumatoid arthritis, auto-inflammatory diseases and diabetes. Blocking IL-1 activity is thought to have few side effects, including minimal to no toxicity or gastrointestinal disturbances.

Thus, further methods and compounds that extend the fertility lifespan or extend and retain the ovarian follicular reserve and improve fertility, are needed.

### SUMMARY OF THE INVENTION

According to a first aspect, the invention relates to an Interleukin-1 (IL-1) inhibitor, specifically, an inhibitor that blocks or inhibits the activity of IL-1, or any composition or formulation thereof, for use in a method of retaining ovarian follicle reserve in a mammalian subject.

The invention provides an IL-1 inhibitor, for use in a method of improving response of the treated mammalian subject to a controlled ovarian hyper stimulation (COH).

Still further, the invention provides an IL-1 inhibitor for use in a method of treating, inhibiting, preventing or ameliorating infertility or related conditions in a mammalian subject.

A further aspect of the invention relates to an ex-vivo method of improving oocyte quality and/or survival. More specifically, the method of the invention may comprise an ex-vivo contacting an oocyte in a cell culture with an effective amount of at least one IL-1 inhibitor or any composition or formulation thereof that blocks or inhibits IL-1 activity, thereby improving oocyte quality and/or survival.

A further aspect of the invention relates to a method for retaining ovarian follicle reserve in a mammalian subject by blocking, inhibiting or reducing the activity of IL-1 in the treated subject. In more specific embodiments, the method of the invention may comprise the step of administering to the subject an effective amount of at least one IL-1 inhibitor or any composition or formulation thereof.

Still further, the method of the invention may be applicable for improving response of the mammalian subject to a COH.

The methods of the invention are particularly applicable for treating, inhibiting, preventing or ameliorating infertility or infertility-related conditions in a mammalian subject. More specifically, the method of the invention may comprise the step of administering to the subject an effective amount of at least one IL-1 inhibitor or any composition or formulation thereof.

A further aspect of the invention relates to a kit comprising:

(a) at least one IL-1 inhibitor or any composition or formulation thereof, optionally, in a first dosage form; and

(b) at least one gonadotropin or any compound inducing COH, optionally, in a second dosage form.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A-1E. The ovarian follicular pool in young and aged WT and IL-1α-KO mice.

FIG. 1A. shows the mean total number of follicles in ovaries of IL-1α-deficient (αKO) compared to normal control (WT) mice of the indicated ages (1.5, 2.5 and 12 months).

FIG. 1B. shows representative micrographs of ovarian sections obtained from 2.5 months old WT and IL-1α KO mice.

FIG. 1C. shows the number of ovarian follicles in each developmental stage [PMF (primordial follicles), P (primary follicles), S (secondary follicles), A (antral follicles)] in 1.5 months old WT and IL-1α-KO mice.

FIG. 1D. shows the number of ovarian follicles in each developmental stage [PMF, P, S, A], in 2.5 months old WT and IL-1α-KO mice.

FIG. 1E. shows the number of ovarian follicles in each developmental stage [PMF, P, S, A], in 12 months old and IL-1α-KO mice.

FIG. 2A-2B. IL-1α-deficiency results in an augmented response to gonadotropins.

FIG. 2A. shows oocytes count of ovulated oocytes in the indicated age in WT and IL-1α-KO (α-KO) mice primed with 10 IU of human chorionic gonadotropins [hCG, an luteinizing hormone (LH) analog]48 hours after pregnant mare serum gonadotropin (PMSG, an FSH analog) administration (7 IU).

FIG. 2B. shows oocytes count of ovulated oocytes in 2.5 months old WT, α-KO, IL-1β-deficient (β-KO) and IL-1 receptor deficient (RIKO) strains of mice, treated with gonadotropins as described in FIG. 2A.

FIG. 3A-3B. Follicle stimulating hormone receptor (FSHR) levels in WT and IL-1α-KO mice.

FIG. 3A. shows the follicle stimulating hormone receptor (FSHR) transcript levels in granulosa cells (GCs) of pre-pubertal (1 months) and pubertal (3 months) WT and IL-1α-KO mice.

FIG. 3B. shows FSHR transcript levels in ovaries of WT and IL-1α-KO pubertal mice 24 hours and 48 hours following ovarian stimulation with gonadotropins (PMSG).

FIG. 4A-4B. IL-1α-KO and IL-1β-KO mice have higher serum AMH compared to WT mice.

FIG. 4A. shows the serum Anti-Mullerian Hormone (AMH) levels in IL-1α-KO, IL-1β-KO and WT mice throughout the fertility lifespan.

FIG. 4B. is a western blot analysis showing the ovarian AMH protein levels in 2.5 months old IL-1α-KO and WT mice.

FIG. 5A-5C. IL-1α, IL-1β and IL-1RI mRNA and IL-1α protein are expressed in the ovary of WT mice.

FIG. 5A. is a graph showing the expression of IL-1α mRNA in the ovary of WT mice during aging (2.5 months to 20 months).

FIG. 5B. is a PCR analysis of RNA extracted from ovaries of WT and IL-1α-KO (αKO) mice and from GCs and GV oocytes of WT mice for detection of IL-1α (α, 125 bp), IL-1β (b, 163 bp) and IL-1RI (R, 368 bp) mRNA transcripts. GAPDH (G, 536 bp) is indicated as internal control.

FIG. 5C. is a western blot of a protein lysate of ovaries from WT and IL-1α-KO mice incubated with anti-IL-1α antibody.

FIG. 6A-6I. IL-1α protein is expressed in GCs and oocytes of WT ovaries.

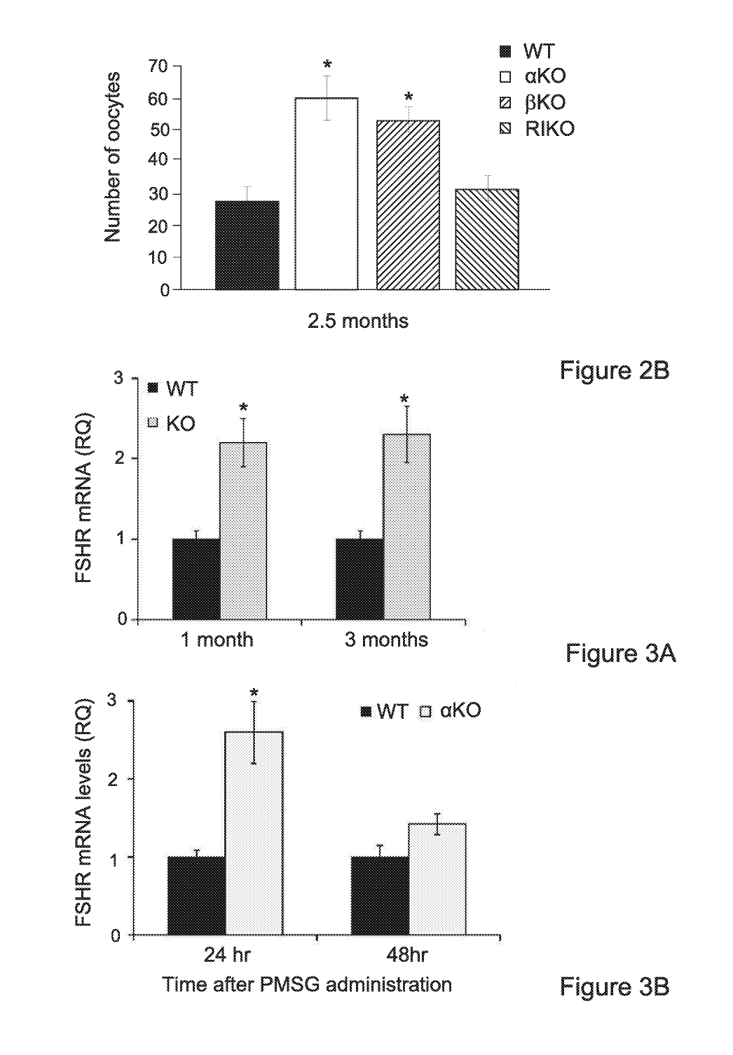

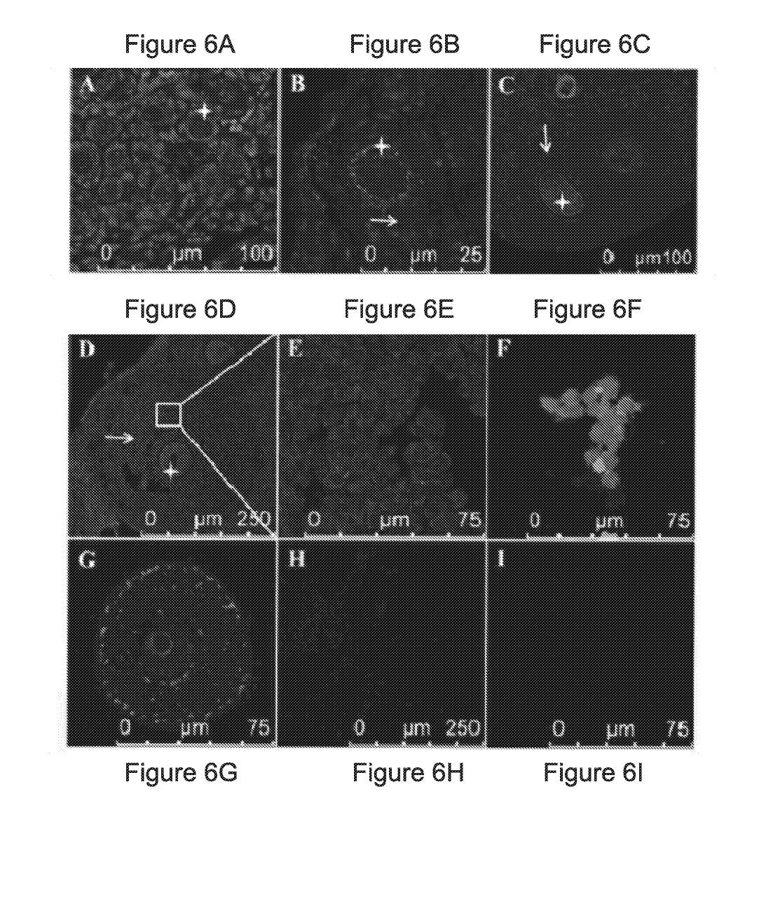

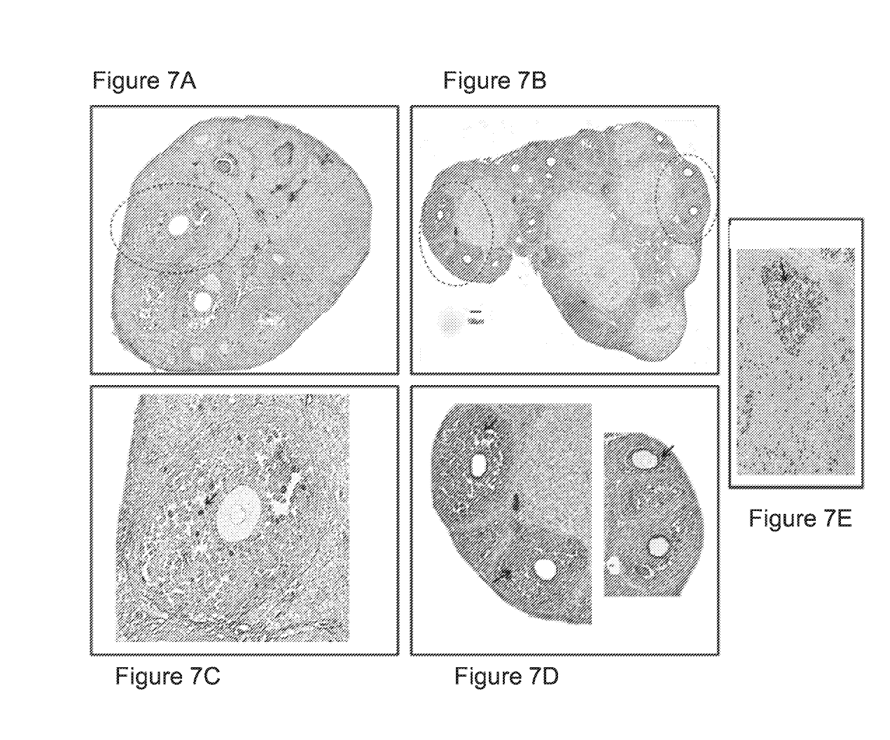

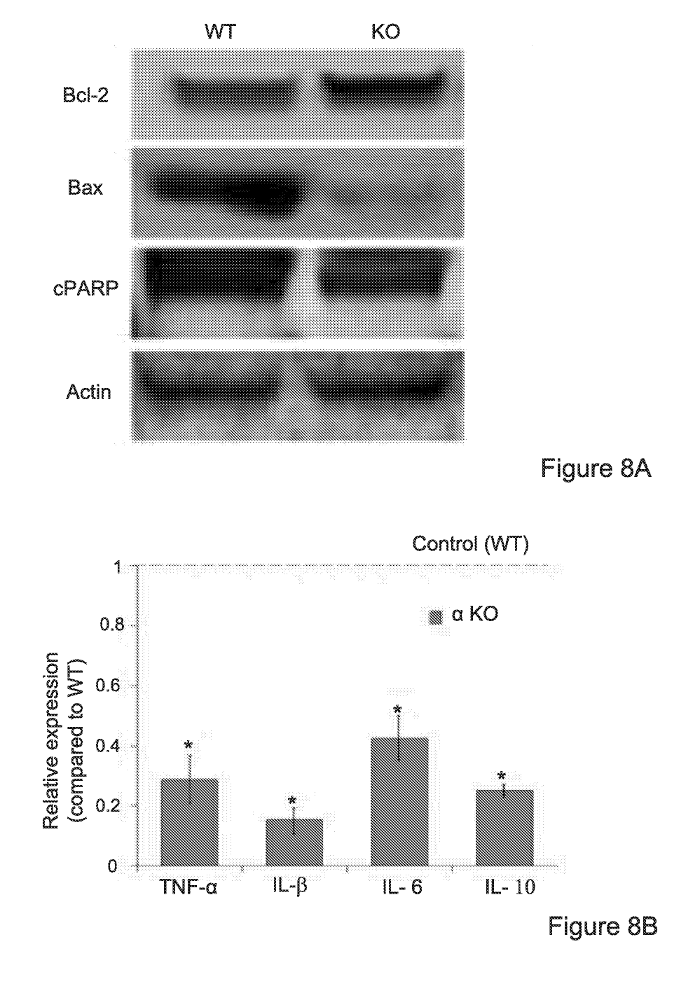

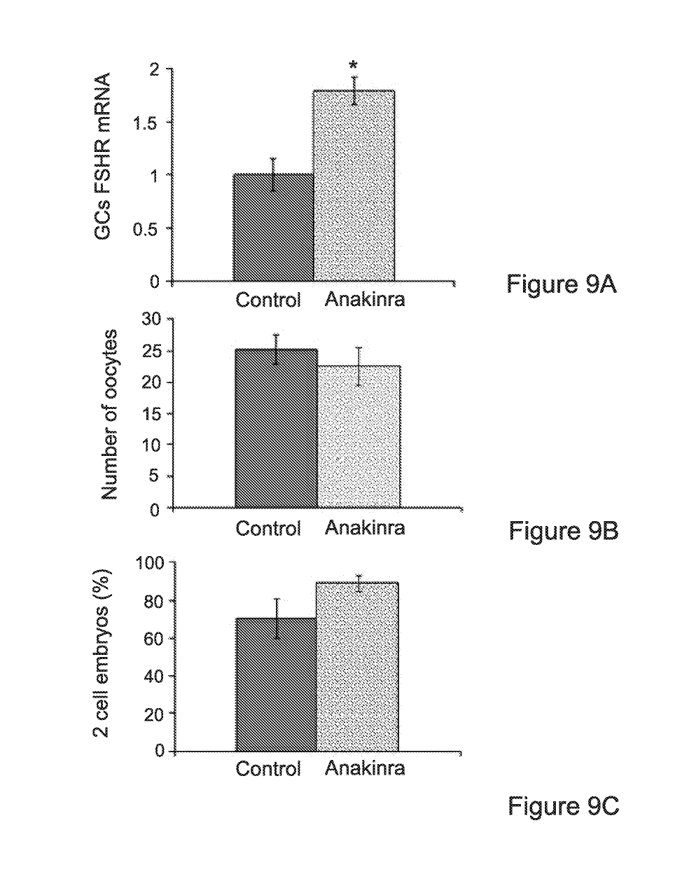

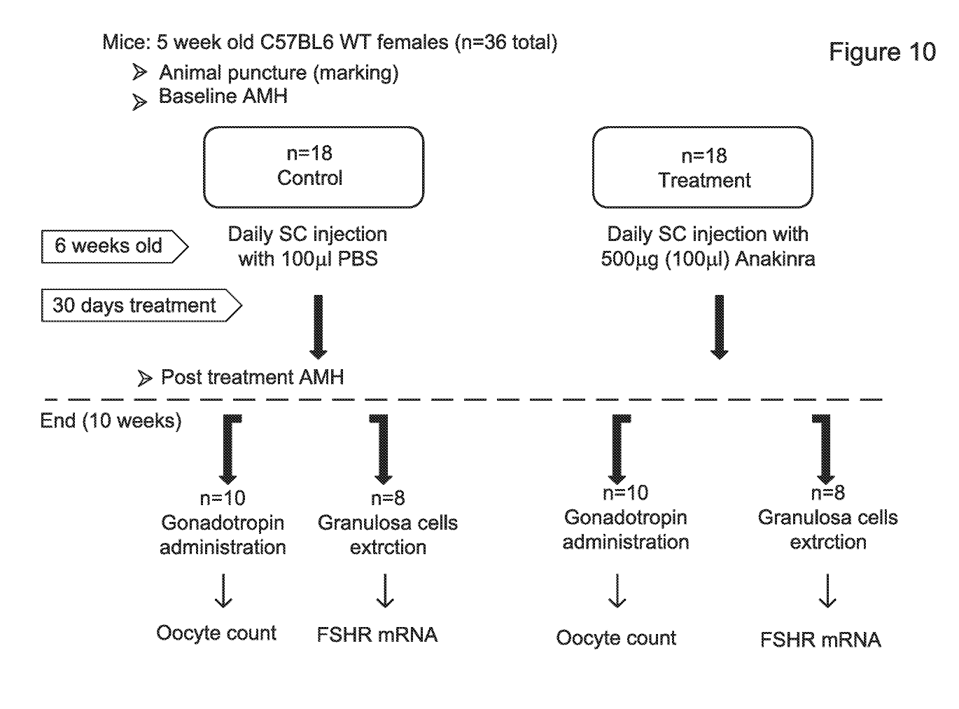

The figure shows ovarian paraffin sections of WT mice stained for IL-1α protein. Paraffin embedded ovarian sections from 6 days (FIG. 6A) and 2.5 month old (FIGS. 6B-6E) WT mice and isolated primary GCs (FIG. 6F) and GV oocytes (FIG. 6G) from 2.5 month old WT mice. Sections were stained with anti-IL-1α antibody (red) and Hoechst (blue) for DNA labeling. IL-1α is detected in the cytoplasm of GCs (arrows) and oocytes (asterisk) throughout follicular development. Negative control using secondary antibody alone is demonstrated in micrographs FIG. 6H (paraffin embedded ovarian section) and FIG. 6I (isolated oocyte). Scale bar is indicated at each micrograph.

FIG. 7A-7E. Increased proliferation of GCs in ovaries of IL-1α-deficient mice.

The figure shows ovarian paraffin sections of 2.5 months old WT (FIGS. 7A, 7C) and IL-1α-KO (FIGS. 7B, 7D) mice stained for nuclear molecular structures that are exclusive for proliferating cells (immunostaning with the Ki-67 antibody). A larger magnification of the indicated areas in FIGS. 7A and 7B are presented in FIGS. 7C and 7D, respectively. Ki-67 staining is evident in follicular GCs (arrows). Positive control of Ki-67 staining is shown in micrographs (FIG. 7E, tonsil). The scale bar (100 micrometer) indicated in the micrograph of FIG. 7B relates to the micrographs in FIGS. 7A and 7B.

FIG. 8A-8B. The expression of apoptotic proteins and inflammation related mRNA levels are lower in ovaries from IL-1α-KO compared to WT mice.

FIG. 8A. shows a western blot of protein lysates of ovaries from 2.5 months old WT and IL-1α-KO mice for the detection of Bcl-2, Bax and the cleaved form of PARP (cPARP). Actin is used as a loading control.

FIG. 8B. shows quantitative polymerase chain reaction (qPCR) analysis for the detection of TNFα, IL-6, IL-1β and IL-10 from RNA extracted from ovaries of 2.5 month old WT and IL-1α-KO mice.

FIG. 9A-9C. IL-1 blockade with Anakinra results in higher FSHR transcripts in mouse GCs, no effect on ovulation, but a slightly improved fertilization rate.

The figure shows results of experiments using therapeutic methods provided by the invention, specifically administering an antagonist to IL-1 (recombinant human IL-1Ra, Anakinra) to WT mice.

FIG. 9A. shows the expression of the FSHR transcript in GCs of mice treated with Anakinra vs. untreated control.

FIG. 9B. shows the number of oocytes retrieved following stimulation with gonadotropins, in WT mice treated with Anakinra vs. untreated control.

FIG. 9C. shows the number of 2 cell embryos in WT mice treated with Anakinra vs. untreated control.

FIG. 10. Therapeutic methods.

The figure shows an outline of studies using therapeutic methods provided by the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The interleukin (IL)-1 family-members are among the most potent molecules of the innate immune system. The two major prototypic agonist cytokines in this family, IL-1α and IL-1β, induce the expression of a variety of pro-inflammatory genes upon binding to IL-1 receptor type 1 (IL-1R1).

Both IL-1α and IL-1β are synthesized as precursors (31 kDa) and are processed to mature forms (17 kDa) via specific cellular proteases. IL-1β is generated upon inflammatory signals and is only active as a mature secreted protein after cleavage by caspase-1. In contrast, IL-1α exerts its effects both in the mature and the precursor forms when binding to IL-1R1. IL-1α is constitutively expressed in the cytosol of epithelial cells, keratinocytes and fibroblasts and is up regulated during inflammation. IL-1α belongs to a newly recognized group of dual-function cytokines that play a role in the nucleus where it affects transcription, apart from their extracellular, receptor-mediated effects.

The mammalian ovary expresses IL-1. It remains unclear regarding the exact nature of the cellular compartments and the time points during the hormonal cycle at which IL-1α and IL-1β are expressed. It is well recognized that inflammation is a hallmark of many processes in reproductive physiology including ovulation, menstruation and implantation.

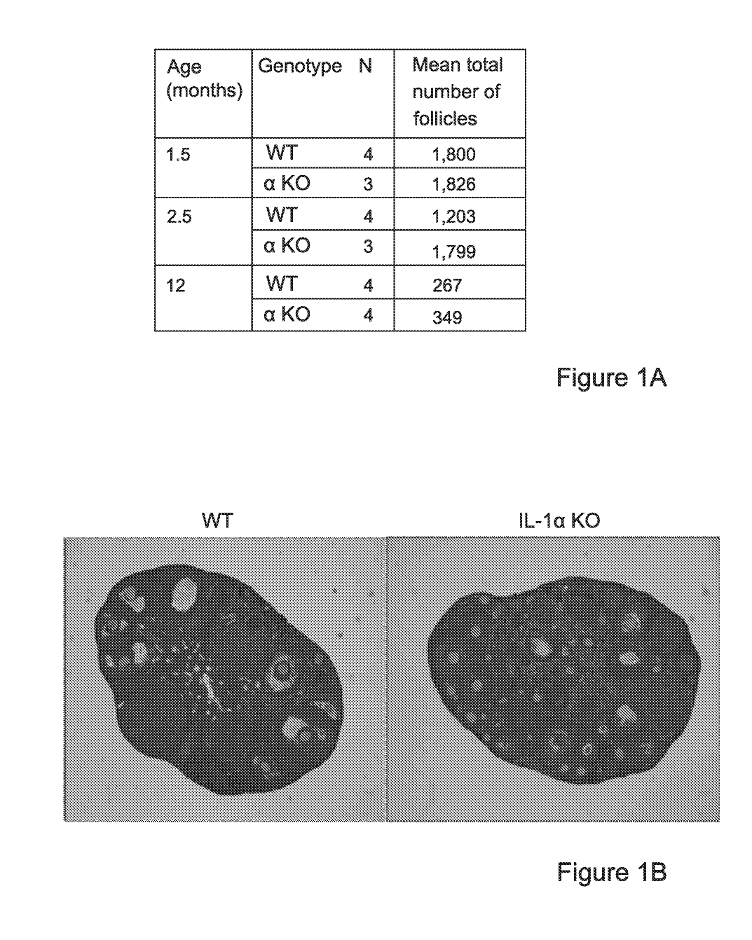

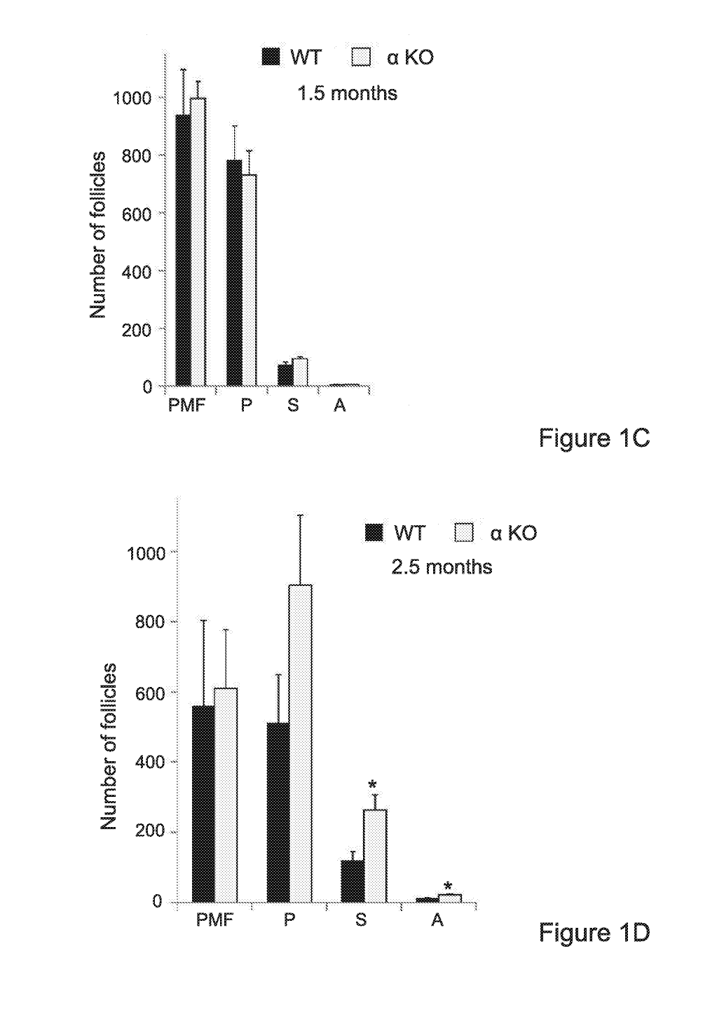

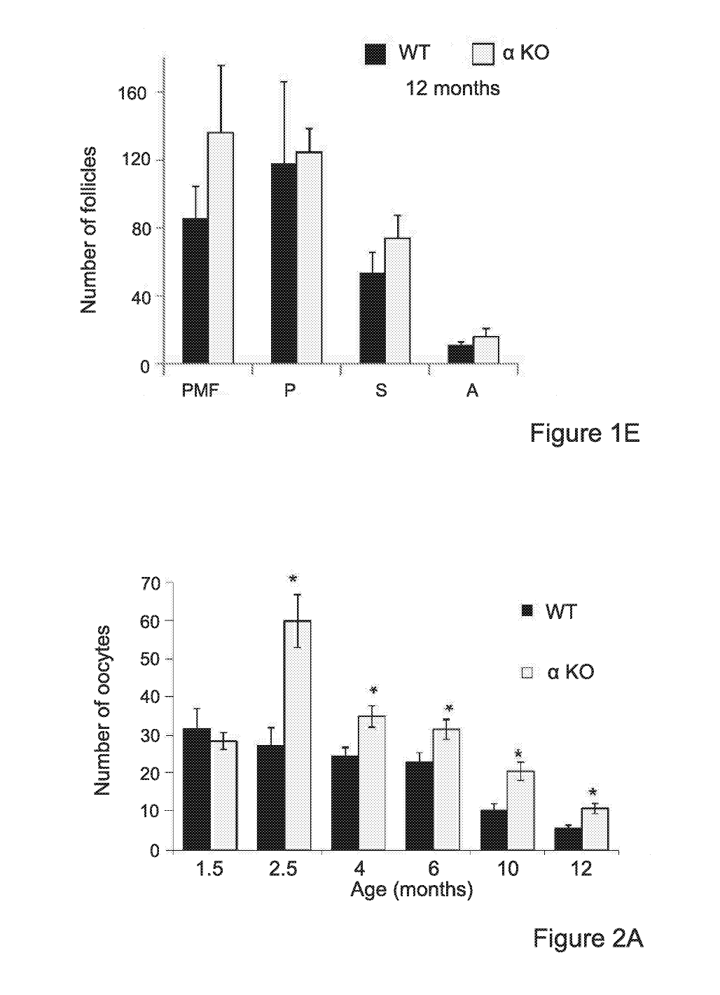

The present invention demonstrates that gene deletion of IL-1α results in a higher pregnancy rate and higher litter size in aged mice. Importantly, the reproductive advantage of IL-1α-KO compared to WT mice is first manifested at 2.5 months of age and lasts up to advanced age. The similar ovarian follicular pool 1.5 months after birth shown in FIG. 1, confirmed previous reports that IL-1α-KO and WT mice are endowed with a comparable number of germ cells. However, the invention surprisingly shows that 2.5 months after birth, ovaries from IL-1α-KO mice contain a significantly higher number of growing follicles compared to WT mice. The dramatically increased response to gonadotropins at 2.5 months and up to one year also supports a higher number of growing follicles in IL-1α-KO compared to WT.

The absence of IL-1, specifically of IL-1α, may therefore increase and enhance retention of the ovarian follicular reserve, specifically in subjects at advanced reproductive age.

Thus, according to a first aspect, the invention relates to an Interleukin-1 (IL-1) inhibitor, specifically, an inhibitor that blocks or inhibits the activity of IL-1, or any composition or formulation thereof, for use in a method of retaining ovarian follicle reserve in a mammalian subject.

The term “retaining” as used herein is meant keeping, maintaining, reserving, and preserving the amount and quality of the ovarian follicular reserve. More specifically, the invention contemplates uses of IL-1 inhibitors and blockers for retaining and maintaining the number and quality of ovarian follicles of any stage, increasing the survival, viability and functionality and preventing apoptosis, elimination and death of the ovarian follicles. This process may also result in improvement of oocyte yield, increased fertilization rate and implementation rate.

The terms “increase”, “elevation”, “enhancement” or “elevation” as referred to herein, relate to the enhancement and increase of the ovarian follicular reserve of a subject, specifically, a mammalian subject, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, as compared to a non-treated subject. With regards to the above, it is to be understood that, where provided, percentage values such as, for example, 10%, 50%, 120%, 500%, etc., are interchangeable with “fold change” values, i.e., 0.1, 0.5, 1.2, 5, 10, 20, 30, 40, 50, 60, 70, 80, 90, 100 etc., respectively.

“Ovarian reserve” as used herein is used to determine the ovarian capacity to provide oocytes that are capable of fertilization resulting in a healthy and successful pregnancy. As will be indicated herein, this term encompasses the quantity as well as quality of ovarian follicles at any stage in a subject.

More specifically, the present invention contemplates reservation, preservation and retention of ovarian follicles in various maturational stages that are important for fertility and reproductive capacity of female mammals, including humans. In other words, the methods and uses of IL-1 inhibitor/s contemplated by the present invention are applicable to the process of folliculogenesis and to various clinical interventions into this process. Under the term ovarian follicle is meant the anatomical structure containing a single primary oocyte (immature ovum) surrounded by a densely packed shell of granulosa cells which numbers increase directly in response to circulating gonadotropin, and further a thin layer of extracellular matrix termed the follicular basement membrane or basal lamina (also fibro-vascular coat) and the layers theca interna and theca externa.

Folliculogenesis describes the progression of a number of small primordial follicles (dormant or resting follicles) into large pre-ovulatory follicles (fully formed differentiated follicles) that enter the menstrual cycle. The number of immature primordial follicles in the ovaries is determined at birth. During post-pubescent follicular development, primordial follicles that have started developing undergo a series of critical changes in character, both histologically and hormonally.

Two-thirds of the way through this process, the follicles have transitioned to tertiary, or antral, follicles. At this stage in development, they become dependent on body hormones causing a substantial increase in their growth rate.

Post-pubertal ovaries are characterized by a heterogeneous population of follicles distinct by size and morphology, including primordial follicles (of about 0.03-0.05 mm in diameter with only one layer of flat granulosa cells), primary follicles (of almost 0.1 mm containing mitotic cells, cuboidal granulosa cells), secondary follicles (of about 0.2 mm with multiple layers of granulosa cells and the presence of theca), tertiary follicles (class 6-10 mm, class 7-16 mm, class 8-20 mm, have antrum, i.e. antral follicles, and no further cell differentiation and no novel progress), and ultimately a preovulatory follicle (destined for ovulation). Roughly a week before the midpoint of the menstrual cycle in one ovary, a dominant follicle develops (i.e. Graffian follicle) that grows faster than the other tertiary follicles (up to 25 mm) and becomes ready for ovulation. The other non-dominant follicles will eventually end in death. The follicular phase, also known as proliferative phase, begins on Day 1 of the menstrual cycle (the period starts) and ends when luteinizing hormone peaks and ovulation occurs. In the early follicular phase, after menstrual flow has ended, the lining of the uterus is at its most thin and estrogen and progesterone are at their lowest level. Later in the follicular phase, proliferation (or thickening) of the uterine lining occurs (preparation for a possible pregnancy). The follicular phase typically lasts about 14 days. The luteal phase begins when the follicular phase ends.

Thus, one important application of the present invention is for preservation and retention of follicles in post-pubertal ovaries of a female mammal. More specifically, for preservation of post-pubertal ovarian follicles, includes primordial follicles, primary, secondary, early and late tertiary, antral follicles, and preovulatory follicles. For the purpose of the present invention, in humans puberty is defined at around 12 years of age. As detailed above, sexual maturation, or “puberty” comprises the physiological and behavioral changes that occur during the transition from juvenile life into reproductive competence.

In some embodiments, the invention provides the IL-1 inhibitor used by the invention or any composition or formulation thereof as herein described, wherein said blocking or inhibition of IL-1 results in a restriction, retardation, reduction, decrease, attenuation, moderation, suppression, inhibition, blocking, attenuation, elimination, repression, weakening or diminishing of the expression, activity, signaling or stability of IL-1 by at least about 1%-100%, about 5%-95%, about 10%-90%, about 15%-85%, about 20%-80%, about 25%-75%, about 30%-70%, about 35%-65%, about 40%-60% or about 45%-55%. Said restriction, retardation, reduction, decrease or diminishing of a process, a phenomenon or a phenotype may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

In some embodiments, blocking or inhibition of IL-1 in said subject, by the IL-1 inhibitor of the invention may lead to reduction in ovarian follicular atresia thereby retaining ovarian follicle reserve in the subject.

Follicular atresia as used herein is the breakdown of the ovarian follicles, which consist of an oocyte surrounded by granulosa cells and internal and external theca cells.

Regardless of the actual mechanism underlying the effect of IL-1 inhibition on follicular retention, it is further contemplated relying on the presently provided examples that the outcomes of IL-1 inhibition are evident on the level of follicular number and reduction of apoptosis, in other words reduction of follicular atresia.

In the process of folliculogenesis, while atresia is the customary fate of a follicle, ovulation represents an exceptional destiny. Although follicular atresia is continuous from the 20^{th} fetal week until almost all follicles are gone by about 50 years of age (in humans), there are three periods in which atresia is more rapid. The largest regression in follicles occurs immediately after the 20^{th} fetal week when the maximum number of around seven million primordial follicles (per ovary) is reached. Immediately following birth, a further short period of accelerated decline takes place. The third, temporally longest period of increased decline, takes place during puberty (begins at the menarche).

Thus, in some embodiments, the present invention is particularly applicable for the reduction of follicular atresia in mammals, specifically, female mammals, at the period between the menarche and the menopause, wherein the menarche is defined by first menstrual cycle or bleeding and the menopause as diminution of menstrual bleeding. Due to this particular feature, the present invention enables to achieve reproductive advantage and increased pregnancy rates at a middle and advanced reproductive age, as also demonstrated in Examples I and II.

In some embodiments, the IL-1 inhibitor/s used by the invention reduce, eliminate, attenuate or inhibit follicular atresia at any stage and type of follicle, as described herein above. More specifically, the IL-1 inhibitors of the invention may reduce atresia in resting ovarian follicles, for example, population of resting primordial follicles, or in maturating ovarian follicles of any developmental type, specifically, primary follicles, early secondary, secondary, and any stage of antral follicle. Therefore, in further embodiments, the invention provides IL-1 inhibitors for use in reducing ovarian follicular atresia in a mammalian subject thereby retaining ovarian follicle reserve in said subject.

The terms “inhibition”, “reduction” or “attenuation” as referred to herein, relate to the retardation, restraining or moderation of ovarian follicular atresia in the treated subject. Such reduction includes reduction by any one of about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%.

The inventors further investigated whether IL-1α deficiency affects apoptosis-related pathways in the ovary. Very few papers report on the role of IL-1α in apoptosis and its involvement in apoptosis within the ovary was not yet approached. In the mammalian ovary, more than 99% of follicles are destined to atretic degeneration (an apoptotic process). Follicular atresia is mediated via the engagement of death ligands (e.g. TNFα and Fas) to plasma membrane death receptors, or through the mitochondrial pathway within the cell in which the Bcl-2 family members play an important role. Nevertheless, the molecular mechanisms responsible for the delicate balance to determine life and death of cells in the ovary are not well understood. As demonstrated in Example VII, the inventors found that IL-1α deficiency results in a higher expression of the anti-apoptotic Bcl-2 protein and lower levels of the pro-apoptotic proteins Bax and PARP within the ovary. The Bcl-2 family members Bcl-2 and Bax are found mainly in developing and atretic follicles, respectively. Furthermore, mRNA levels of the cell death inducing cytokine TNFα, which was previously shown to induce apoptosis within the ovary was 3.5 fold lower in IL-1α-KO compared to WT ovaries. Taken together, these findings suggest that IL-1α promotes apoptotic signaling within the ovarian follicles both by the extracellular TNFα pathway and the intracellular mitochondrial pathway. Importantly, IL-1α deficiency resulted in reduced ovarian expression of pro-inflammatory cytokines other than TNFα, such as IL-1β and IL-6. The lower mRNA levels of the anti-inflammatory cytokine IL-10 can be attributed to a decreased necessity to counteract the inflammatory milieu which is attenuated in IL-1α-KO.

Thus, in more specific embodiments, the reduction in ovarian follicle atresia may be a result of at least one of increase in the expression or activity of pro-survival proteins, decrease in the expression or activity of pro-apoptotic proteins and decrease in the expression or activity of pro-inflammatory cytokines, in ovarian cells of said subject.

In more specific embodiments, depletion of IL-1α results in an increase in pro-survival Bcl2 expression, decreased expression of the pro-apoptotic Bax and PARP, and decreased expression of pro-inflammatory cytokines including TNF-α, IL-6 and IL-1β and decreased expression of anti-inflammatory cytokine IL-10. It should be noted that in certain embodiments, depletion of IL-1 may also result in increased expression of other pro-survival proteins, for example, Bcl-xL, Bcl-w, A1/Bfl-1 and Bcl-B/Bcl2L10, decreased expression of other pro-apoptotic proteins such as Bak, Bnip3, Nix/Bnip3L, Bid, Noxa, Puma and Bad, as well as the decreased expression of a family of highly potent and specific proteases, termed caspases (for cysteine-aspartate protease) and decreased expression of pro-inflammatory cytokines.

As used herein the term Bcl-2 pro-survival protein refers to a proto-oncogenic protein known as an apoptosis inhibitor. The Bcl-2 protein forms the basis of a growing family of related proteins collectively denoted herein as Bcl-2 family of proteins. These proteins are known to control apoptotic cell death by the mitochondrial pathway.

As appreciated in the art, the members of the Bcl-2 family are either pro-survival or pro-apoptotic but regardless of their activity, they all share significant sequence and structural homology. Specifically, the Bcl-2 family of proteins is characterized by up to four regions of sequence homology, known as the Bcl-2 homology (BH) domains.

As previously described in the art, the Bcl-2 family of proteins includes three different groups of proteins: the first group is a pro-survival or anti-apoptotic group denoted herein as “Bcl-2 pro-survival proteins”, the second group is a pro-apoptotic group including BAX and BAK; and a third group denoted herein as BH3-only proteins that exhibit a pro-apoptotic activity.

The term “apoptosis” refers to a regulated network of biochemical events which lead to a selective form of cell suicide and is characterized by readily observable morphological and biochemical phenomena. Cells undergoing apoptosis show characteristic morphological and biochemical features. These features include chromatin aggregation or condensation, DNA fragmentation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis.

The terms “inhibition”, “moderation” or “attenuation” as referred to herein, relate to the retardation, restraining or reduction of the pro-apoptotic activity of a Bcl-2 pro-apoptotic family member. Such inhibition may be of about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%. In certain embodiments, such pro-apoptotic Bcl-2 family member may include any one of Bax, Bak, Bnip3, Nix/Bnip3L, Bid, Noxa, Puma, and Bad.

In yet some other embodiments, the invention provides the use of IL-1 inhibitors that lead to an increase or enhancement of Bcl-2 pro-survival proteins. Said increase, induction or elevation of survival may be an increase by about 1% to 99.9%, specifically, about 1% to about 95%, about 5% to 90%, about 10% to 85%, about 15% to 80%, about 20% to 75%, about 25% to 70%, about 30% to 65%, about 35% to 60%, about 40% to 55%, about 45% to 50%. More specifically, about 5%, 10%, 15%, 20%, 25%, 30%, 35%, 40%, 45%, 50%, 55%, 60%, 65%, 70%, 75%, 80%, 85%, 90%, 95% or 99.9%. More specifically, an increase of about 5%, 10%, 15%, 20%, 25%, 30%, 35%, 40%, 45%, 50%, 55%, 60%, 65%, 70%, 75%, 80%, 85%, 90%, 95% or 99% as compared to untreated control.

Still further, as discovered by the invention, mRNA levels of FSHR, which is expressed by growing follicles, were higher in IL-1α-KO compared to WT ovaries 24 hours after PMSG administration. These findings clearly indicate that from 2.5 months onwards, IL-1α-KO mice contain a larger population of viable growing follicles available for PMSG priming.

Therefore, in further embodiments, blocking or inhibition of IL-1 by the inhibitors used by the invention may further results in increased expression of follicular stimulating hormone receptor (FSHR) in cells comprised within ovarian follicles of said subject.

In some embodiments, an increased expression of FSHR in said follicular cells increases, enhances, supports and promotes ovarian follicular maturation.

Still further, in some embodiments, blocking or inhibition of IL-1 may further result in increased ovarian and serum levels of anti-Mullerian hormone (AMH), a putative marker of ovarian reserve.

In this connection, the anti-Müllerian hormone (AMH) refers to the protein encoded by the AMH gene at 19p13.3, its receptors is encoded by the AMHR2 gene on chromosome 12. Apart from other functions, particularly relevant to the present context is that AMH is expressed by granulosa cells of the growing follicles in the ovary and limits the transition of PMF into primary follicles. A number of clinical protocols use AMH as a measure of certain aspects of ovarian function, particularly in assessing conditions such as polycystic ovary syndrome and premature ovarian failure, and also to predict a poor ovarian response in in vitro fertilization (IVF).

As shown by the invention, depletion of IL-1, specifically of IL-1α, results in an increased ovarian follicular reserve. Therefore, the invention contemplates the use of any compound that blocks or inhibits IL-1.

In certain embodiments, the IL-1 inhibitor used by the invention may be an inhibitor that blocks or inhibits at least one of, the activity, expression, stability, post-transcriptional or posttranslational processing of at least one of IL-1α, IL-1β, Interleukin-1 receptor (IL-1R1) or of any molecule participating in signal transduction pathway/s mediated by IL-1. More specifically, the IL-1 inhibitor/s used by the invention may affect the stability of any one of specifically, IL-1α, IL-1β, IL-1R1 or of any molecule participating in signal transduction pathway/s mediated by IL-1. Stability, as used herein reflects the ability of a protein to retain its structural conformation, integrity or its biological activity. In further embodiments, the inhibitors of the invention may affect post transcriptional processing of said proteins. It should be noted that a pre-mRNA molecule undergoes three main modifications including 5′ capping, 3′ polyadenylation, and RNA splicing, which occur in the cell nucleus before the RNA is translated. These modifications affect the stability of the mRNA and thereby the expression of a certain protein. Still further, the IL-1 inhibitor/s may also affect posttranslational modifications and processing of the above-mentioned proteins. More specifically, post translational modifications of amino acids extends the range of functions of the protein by attaching it to other biochemical functional groups (such as acetate, phosphate, various lipids and carbohydrates), changing the chemical nature of an amino acid (e.g. citrullination), or making structural changes (e.g. formation of disulfide bridges). In yet another embodiment, the IL-1 inhibitors of the invention may affect the expression of any of the above-mentioned proteins, specifically, IL-1α, IL-1β, IL-1R1 or of any molecule participating in signal transduction pathway/s mediated by IL-1. Non limiting examples for proteins that participate in IL-1 signaling include protein such as IL-6, IL-8, MCP-1, COX-2, IκBα, IL-1α, IL-1β or MKP-1.

In some specific embodiments, the IL-1 inhibitor used by the invention may be an inhibitor that blocks or inhibits at least one of, the activity, expression, stability, post-transcriptional or posttranslational processing of IL-1α.

When referring to an interleukin 1 (abbreviated IL-1) is meant a small and water-soluble signaling protein belonging to the IL-1 superfamily of interleukin cytokines that play a central role in various immune responses, inflammatory processes and hematopoiesis. As mentioned above, IL-1 is considered the master cytokine of local and systemic inflammation that are induced upon its binding to the type 1 IL-1 receptor (IL-1R1). The naturally occurring endogenous IL-1 receptor antagonist (IL-1Ra) further regulates IL-1 pro-inflammatory activities by competing for IL-1R1 binding sites.

Specifically, under IL-1 is meant the two IL-1 isoforms, IL-1α and IL-1β, both of which are capable of binding to IL-1R1. Under IL-1α (also IL-1A, pro-interleukin-1α or -1A, IL-1F1 or hematopoietin-1) is meant the IL-1α isoform expressed as a precursor that is constitutively present in most cells of a healthy subject. Specifically the human IL-1α is a 271 amino acid precursor weight 30.6 kDa (Acc. Num. P01583, NCBI Ref Seq GI:124297, as denoted by SEQ ID NO. 25) encoded by the gene mapped to the IL-1 gene cluster at the 2q13-14 chromosome (Acc. Num. NC_018913.2, the cDNA is disclosed by NCBI Ref Seq GI:27894329; NM_000575.3, as denoted by SEQ ID NO. 26). IL-1α has been associated with a number of biological activities, among others, an inflammatory response, fever generation, cell proliferation and controlled release of certain cellular factors, and more specifically cellular response to heat, response to copper ions, cytokine-mediated signaling pathway, positive regulation of cell division, positive regulation of cytokine secretion particularly of IL-2 and vascular endothelial growth factor (VEGF), positive regulation of transcription from RNA polymerase II promoter. It should be appreciated that in certain embodiments, the invention further relates to the murine IL-1α as disclosed in NCBI Ref Seq GI:124298; SP:P01582.1, denoted by SEQ ID NO. 46, encoded by cDNA disclosed in NCBI Ref Seq GI: 118130060; NM_010554.4, as denoted by SEQ ID NO. 47.

When referring to IL-1β (also IL-1B, pro-interleukin-1β or 1B IL-1F2 or catabolin) is meant the IL-1β isoform, which unlike IL-1α is not present in health or only at levels not detected by standard assays. The main IL-1β producing cells are monocytes, tissue macrophages and dendritic cells, where it is first synthesized as an inactive precursor that is activated upon stimuli by cleavage with caspase-1, an intracellular cysteine protease. The human IL-1β is a 269 amino acid precursor weight 30.7 kDa (Acc. Num. P01584, NCBI Ref Seq GI:62906858, as denoted by SEQ ID NO. 27) encoded by the same gene cluster (Acc. Num. NC_018913.2, the cDNA is disclosed by NCBI Ref Seq GI:27894305; NM_000576.2, as denoted by SEQ ID NO. 28). IL-1β has been associated with cellular response to drugs, organic substances, mechanical stimuli, apoptotic processes, cell-cell signaling and signal transduction specifically the cytokine-mediated signaling pathway, and more specifically, among others, negative regulation of cell proliferation, glucose transport, lipid catabolic and metabolic processes and MAP kinase activity, and positive regulation of cell division, heterotypic cell-cell adhesion, chemokine biosynthesis, gene expression, histone acetylation and phosphorylation. In yet other embodiments, the invention further relates to the murine IL-1β as disclosed in NCBI Ref Seq GI:124304; SP:P10749.1, denoted by SEQ ID NO. 48, encoded by cDNA disclosed in NCBI Ref Seq GI: 118130747; NM_008361.3, as denoted by SEQ ID NO. 49.

Still further, when the present invention is contemplated it should be understood that IL-1 or IL-1 signaling also involves IL-1 binding receptors IL-1R type I and II. Specifically the IL-1R type I (also IL-1R1, IL-1Rα or -13RA, IL-1RT-1, CD121 antigen-like family member A or CD121A or p80) that is expressed nearly in all tissues has been shown to bind IL-1α and -1β and the natural antagonist IL-1Ra with a high affinity. The human IL-1R type I is a precursor of 569 amino acids weight 65.4 kDa (Acc. Num. P14778, NCBI Ref Seq GI:124308, as denoted by SEQ ID NO. 29) encoded by the same gene cluster, specifically, encoded by cDNA disclosed in NCBI Ref Seq GI:569026721; NM_000877.3 (transcript variant 1), as denoted by SEQ ID NO. 30. It should be noted that in specific embodiments, the invention further relates to IL1R1 transcript variant 2, mRNA disclosed in NCBI Ref Seq GI:569026722; NM_001288706.1, as denoted by SEQ ID NO. 31. In contrast, the IL-1R type II (also IL-1R2, IL-1R β or -1RB, IL-1T2, IL-1R2c, CD121B or antigen CDw121b) has been shown to bind IL-1α and IL-1β and IL-1R1, thereby acting as a decoy receptor that inhibits the activity of its ligands. The human IL-1R type II is a 398 amino acid precursor weight 45.4 kDa (Acc. Num. P27930, NCBI Ref Seq GI: 124310, as denoted by SEQ ID NO. 32) encoded at the same gene region. The invention further relates to human Interleukin-1 receptor type 2 isoform 2 precursor, short isoform disclosed in NCBI Ref Seq GI:387527984; NP_001248348.1 as denoted by SEQ ID NO. 33. Still further, the invention relates to IL1R2 transcript variant 1, mRNA disclosed in NCBI Ref Seq GI:27894332; NM_004633.3, as denoted by SEQ ID NO. 34, as well as to IL1R2 transcript variant 3, mRNA disclosed in NCBI Ref Seq GI:387527983; NM_001261419.1, as denoted by SEQ ID NO. 35.

It should be appreciated that in certain embodiments, the invention further relates to the murine IL-1R type I, as disclosed in NCBI Ref Seq GI:124309; SP:P13504.1, denoted by SEQ ID NO. 50, encoded by transcript variant 1 cDNA disclosed in NCBI Ref Seq GI:118130781; NM_0083 0.2, as denoted by SEQ ID NO. 51, as well as to transcript variant 2 cDNA disclosed in NCBI Ref Seq GI:183396775; NM_001123382.1, as denoted by SEQ ID NO. 52.

Still further, the invention relates to the mouse IL-1R type II, as disclosed in NCBI Ref Seq GI:124311; SP:P27931.1, as denoted by SEQ ID NO. 53, encoded by the cDNA disclosed in NCBI Ref Seq GI:76253864; NM_010555.4, as denoted by SEQ ID NO. 54.

Further in this connection, the IL-1 or IL-1 mediated signaling involves various IL-1R1 or -1R2 associated kinases (IRAKs) and accessory or binding proteins that are critical for IL-1R1 or -1R2 activity, most notably the Myeloid Differentiation Primary Response 88 (MYD88) adapter protein and IRAK4 and IRAK2, all of which are encompassed by the present invention. In other words, the present invention contemplates application of any inhibitor or blocker of IL-1 capable of interfering with the IL-1 mediated signaling. Such inhibitor may be directed specifically against IL-1α, IL-1β, IL-1R1 or against any molecule participating in signal transduction pathway/s mediated by IL-1.

An important endogenous modulator of IL-1 mediated signaling is the natural IL-1 inhibitor (IL-1Ra or IL-1RA and also IL-1RN, IL-1F3, Intracellular Interleukin 1 (1c1L-1ra), IL-1 type II, MVCD4, DIRA, Anakinra), which is capable of inhibiting the activities of both, IL-1α and IL-1 and thereby of modulating a variety of related immune and inflammatory functions. Most notably, IL-1Ra has been shown to inhibit IL-1 by binding to IL-1R1 with even greater affinity than the IL-1R2 decoy receptor and by preventing association of the co-receptor IL-1RAP required for the IL-1 signaling. The human IL-1Ra is a 77 amino acid precursor weight 20.0 kDa (Acc. Num. P18510, NCBI Ref Seq GI (precursor isoform 1), as denoted by SEQ ID NO. 36) belonging to the same IL-1 gene cluster, encoded by transcript variant 1 cDNA disclosed in NCBI Ref Seq GI:296010861; NM_173842.2, as denoted by SEQ ID NO. 40. It should be noted that the invention further encompass the human IL-1R antagonist protein isoform 2, disclosed in NCBI Ref Seq GI:27894317; NP_776213.1, as denoted by SEQ ID NO. 37, isoform 3 disclosed in NCBI Ref Seq GI: 10835147; NP_000568.1, as denoted by SEQ ID NO. 38 and isoform 4 disclosed in NCBI Ref Seq GI:27894321; NP_776215.1, as denoted by SEQ ID NO. 39, as well as to the transcript variant 2, mRNA (cDNA) disclosed in NCBI Ref Seq GI:296010856; NM_173841.2, as denoted by SEQ ID NO. 41, the transcript variant 3 cDNA disclosed in NCBI Ref Seq GI:296010855; NM_000577.4, as denoted by SEQ ID NO. 42 and the transcript variant 4 cDNA disclosed in NCBI Ref Seq GI:296010857; NM_173843.2, as denoted by SEQ ID NO. 43.

Still further, the invention further relates to the murine IL-1ra as disclosed in NCBI Ref Seq GI:124313; SP:25085.1, denoted by SEQ ID NO. 55, murine IL-1ra isoform 1, disclosed in NCBI Ref Seq GI:13624317; NP_112444.1, as denoted by SEQ ID NO. 56, murine IL-1ra isoform 2, disclosed in NCBI Ref Seq GI:89257344; NP_001034790.1, as denoted by SEQ ID NO. 57 and the murine IL-1ra isoform 3, disclosed in NCBI Ref Seq GI:227116259; NP_001153034.1, as denoted by SEQ ID NO. 58. The invention further relates to murine IL-1ra transcript variant 1 mRNA (cDNA) disclosed in NCBI Ref Seq GI:227116256; NM_031167.5, as denoted by SEQ ID NO. 59, transcript variant 2, disclosed in NCBI Ref Seq GI:227116257; NM_001039701.3, as denoted by SEQ ID NO. 60 and to transcript variant 3 disclosed in NCBI Ref Seq GI:227116258; NM_001159562.1, as denoted by SEQ ID NO. 61.

Further in this connection, an important IL-1Rα paralog is IL-36α (also IL-36A or Interleukin 1 Family Member 6 or Epsilon also IL-1ε or IL-1E, IL-1F6 and FIL1E), which GO (gene onthology) annotations include IL-1R receptor binding and cytokine activity. The human IL-36A has been identified with a 158 amino acids protein product weight 17.6 kDa (Acc. Num. NCBI Ref Seq GI:25008601; SP:Q9UHA7.1, as denoted by SEQ ID NO. 44) and the IL36A, mRNA (cDNA) disclosed in NCBI Ref Seq GI:7657091; NM_014440.1, as denoted by SEQ ID NO. 45. Still further, the invention relates to the murine IL-36A disclosed in NCBI Ref Seq GI:9506601; NP_062323.1, as denoted by SEQ ID NO. 62, as well as to the murine IL-36A mRNA disclosed in NCBI Ref Seq GI: 133892651; NM_019450.3, as denoted by SEQ ID NO. 63.

Still further, yet another interleukin IL-4 (also Lymphocyte Stimulatory Factor 1, B Cell Stimulatory or Growth Factor 1, BSF-1 and BCGF-1 and also Binetrakin or Pitrakinra) has been known to both to suppress the expression of IL-1, particularly of IL-1β, while concomitantly to enhance the synthesis of IL-1Ra mRNA and protein, and in this manner to antagonize IL-1 activity. The human IL-4 is a 153 amino acid precursor weight 17.4 kDa (Acc. Num. P05112) and its encoding gene mapped to 5q31.1.

As in its main application, the present invention aims at inhibiting IL-1 activity for the purpose of retaining or preserving the ovarian follicular reserve in a mammal, it is thus conceivable that when practicing the present invention the above factors participating in IL-1 signaling, their analogs, agonists and antagonists, synthetic or natural, may be applicable for this purpose.

In specific embodiments, the present invention contemplates use of variants or derivatives, specifically mutation harboring and polymorphism harboring variants, splice variants, alternative transcription products and protein isoforms of the above-mentioned proteins and genes, which may have enhanced IL-1 inhibiting effects. For example, a number of common polymorphisms in IL-1, IL-1R and particularly IL-1Ra have been associated with clinical indices of immune response and inflammation.

In this context are also applicable homologues and orthologs of the above-mentioned proteins and genes, particularly of a mammalian origin, as well as derivatives or functional derivatives of the same proteins and genes, particularly those associated with enhanced IL-1 inhibiting effects. Said derivatives, nucleotide- or protein-based may have only partial homologies or constitute fragments of the above proteins and genes related to an improved desirable biological activity.

Protein and peptide derivatives may have additional secondary modifications, e.g. glycosylated, acylated, amidated or esterified to improve the desirable biological activity. Further, they may harbor non-naturally occurring amino acids, analogs and mimetics to improve biological activity or to facilitate their production and formulation in a clinically applicable form. Still further, said derivatives maybe produced in a form of fusion proteins or peptides.

All the proposed variants, homologues and derivatives may be prepared by recombinant DNA technologies or large scale peptide synthesis methods using protocols that are known and practiced by those skilled in the art. A number of such reagents have been already produced and implemented for the treatment of rheumatoid arthritis, osteoarthritis, diabetes, ischemic and kidney damage and certain other chronic and genetic inflammatory conditions, but for the clinical purpose as defined by the present invention.

In more specific embodiments, IL-1 inhibitor/s applicable for the invention may be any synthetic or natural inhibitor, for example, any protein compound, peptidomimetic compound, nucleic acid compound, a small molecule or any combinations thereof.

In certain embodiments, the IL-1 inhibitors applicable for the invention may be a natural or synthetic nucleic acid compounds. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties, and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. It should be appreciated that the invention may further refer to polyribonucleotide. The term “polyribonucleotide” refers to a polynucleotide comprising two or more modified or unmodified ribonucleotides and/or their analogs. The term “polyribonucleotide” is used interchangeably with the term “oligoribonucleotide”. In more specific embodiments a nucleic acid molecule according to the invention may be an iRNA molecule, more specifically, dsRNA molecule.

Still further, in other embodiments, the IL-1 inhibitors used by the invention may be natural or synthetic protein or polypeptide-base compounds. The term “polypeptide” as used herein refers to amino acid residues, connected by peptide bonds. A polypeptide sequence is generally reported from the N-terminal end containing free amino group to the C-terminal end containing free carboxyl group. A polypeptide may also be termed amino acid sequence, peptide, or protein and can be modified, for example, by manosylation, glycosylation, amidation, carboxylation or phosphorylation.

As also mentioned herein before, derivative of the polypeptides are also encompassed by the present invention. Derivatives are meant to include polypeptides that differ in one or more amino acids in the overall sequence, polypeptides that have deletions, substitutions, inversions or additions. It is appreciated that these polypeptide modifications and polypeptide derivatives must not alter the activity of the original polypeptides.

It should be noted that the polypeptides according to the invention can be produced synthetically, or by recombinant DNA technology. Methods for producing polypeptides peptides are well known in the art.

In more specific embodiments, IL-1 inhibitor/s applicable for the invention may be nucleic acid-based compounds that specifically target IL-1α, IL-1β, IL-1R1 or any molecule participating in signal transduction pathway/s mediated by IL-1. Such inhibitors may specifically block or inhibit the expression of the target molecule, for example by means of RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene RNA sequence.

As used herein, the term “RNA silencing agent” refers to an RNA which is capable of inhibiting or “silencing” the expression of a target gene, for example, IL-1α, IL-1β, IL-1R1 or any molecule participating in signal transduction pathway/s mediated by IL-1. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

Thus, in some further embodiments, the nucleic acid IL-1 inhibitor of the invention may be a ribonucleic acid (RNA) selected from the group consisting of an antisense RNA, a single-stranded RNA (ssRNA), a Ribozyme and a double-stranded RNA (dsRNA).

More specifically, the nucleic acid based IL-1 inhibitor used by the invention may be an “antisense RNA”, which is an ssRNA molecule that is complementary to an mRNA strand. Antisense RNA may be introduced into a cell to inhibit the translation of a complementary mRNA by base-pairing to it and physically obstructing the translation machinery.

Another agent capable of down regulating a target gene product, for example, IL-1α, IL-1β, IL-1R1 or any molecule participating in signal transduction pathway/s mediated by IL-1 may be a Ribozyme molecule capable of specifically cleaving an mRNA transcript encoding said target gene product. Ribozymes are being increasingly used for the sequence-specific inhibition of gene expression by the cleavage of mRNAs encoding proteins of interest. The possibility of designing Ribozymes to cleave any specific target RNA, has rendered them valuable tools in both basic research and therapeutic applications.

An additional method of down regulating the expression of a target gene product in cells may be via triplex forming oligonuclotides (TFOs). Recent studies have shown that TFOs can be designed which can recognize and bind to polypurine/polypirimidine regions in double-stranded helical DNA in a sequence-specific manner. Triplex-forming oligonucleotides may range from about 15 to about 100 bp in length. Formation of the triple helical structure with the target DNA induces steric and functional changes, blocking transcription initiation and elongation, allowing the introduction of desired sequence changes in the endogenous DNA and resulting in the specific down regulation of gene expression.

It should be further appreciated that other agents capable of down regulating a target gene product, for example, IL-1α, IL-1β, IL-1R1 or any molecule participating in signal transduction pathway/s mediated by IL-1, may be DNAzyme molecules. DNAzymes are single-stranded polynucleotides which are capable of cleaving both single and double stranded target sequences, specifically, an mRNA transcript or DNA sequence of the target polypeptide.

In yet further embodiments, the nucleic acid IL-1 inhibitor applicable for the invention may be a dsRNA molecule participating in RNA interference. RNA interference (RNAi), as indicated above, is a general conserved eukaryotic pathway which down regulates gene expression in a sequence specific manner.

More specifically, the dsRNA encompassed by the invention may be selected from the group consisting of small interfering RNA (siRNA), MicroRNA (miRNA), short hairpin RNA (shRNA) and PIWI interacting RNAs (piRNAs). As known in the art, RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs containing a sequence complementary to one of the siRNA strands. One of the strands of the double-stranded siRNA molecule comprises a nucleotide sequence that is complementary to a nucleotide sequence of the target gene, or a portion thereof, and the second strand of the double-stranded siRNA molecule comprises a nucleotide sequence substantially similar to the nucleotide sequence of the target gene, or a portion thereof.

The polynucleotide down-regulating agents that may serve as IL-1 inhibitors by the present invention may be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

It will be appreciated that a polynucleotide IL-1 inhibitor agent used by the methods of the present invention may be provided per se, or as a nucleic acid construct comprising a nucleic acid sequence encoding the polynucleotide agent. Typically, the nucleic acid construct comprises a promoter sequence which is functional in the host cell.

It should be appreciated that the invention further contemplates the use of IL-1 inhibitor/s that a oligonucleotide or oligonucleotide analogues, for example, LNA, PNA, INA and any mixtures, hybrids or modifications thereof. When used in the present context, the terms “locked nucleic acid monomer”, “locked nucleic acid residue”, “LNA monomer” or “LNA residue” refer to a bicyclic nucleotide analogue. An INA is an oligonucleotide or oligonucleotide analogue comprising one or more intercalator pseudonucleotide (IPN) molecules. Peptide nucleic acid (PNA) is an artificially synthesized polymer similar to DNA or RNA and is used in biological research and medical treatments. PNA is not known to occur naturally.

In certain embodiments, the invention provides a nucleic acid based inhibitor that specifically targets IL-1α, thereby inhibiting and blocking IL-1 activity or signaling. Such IL-1α specific nucleic acid-based inhibitor may be for example any one of LNAs, PNAs, nucleic acids encoding a natural or recombinant IL-1 antagonist, or inhibitor, expression vectors/cassettes comprising the same, a ribonucleic acid (RNA) selected from the group consisting of a double-stranded RNA (dsRNA), an antisense RNA, a single-stranded RNA (ssRNA) and a Ribozyme.

Still further, the invention contemplates the use of an IL-1 inhibitor that may be a proteineous, or protein-based compound.

It should be noted that the IL-1 inhibitor used by the invention may be an antibody. Specifically, an antibody directed against any one of IL-1α, IL-1β, IL-1R1 or against any molecule participating in signal transduction pathway/s mediated by IL-1. The term “antibody” as used in this invention includes whole antibody molecules as well as functional fragments thereof, such as Fab, F(ab′)2, and Fv that are capable of binding with antigenic portions of the target polypeptide, i.e. IL-1α, IL-1β, IL-1R1 or any molecule participating in signal transduction pathway/s mediated by IL-1. The antibody may be monospecific, e.g., a monoclonal antibody, or antigen-binding fragment thereof, or bi-specific. The term “monospecific antibody” refers to an antibody that displays a single binding specificity and affinity for a particular target, e.g., epitope. The term “bi-specific antibody” refers to an antibody that displays a dual binding specificity and affinity for, and targeted against two epitopes of the same or of different molecules. This term includes a “monoclonal antibody” or “monoclonal antibody composition”, which as used herein, refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, IgM or subtypes thereof. The antibody can be conjugated to a functional moiety (e.g., a compound which has a biological or chemical function. The antibody used as an IL-1 inhibitor or blocker by the invention, interacts with a polypeptide that is IL-1α, IL-1β or IL-1R1 or any molecule participating in signal transduction pathway/s mediated by IL-1, with high affinity and specificity, thereby blocking the activity and function of IL-1.

As noted above, the term “antibody” also encompasses antigen-binding fragments of an antibody. The term “antigen-binding fragment” of an antibody (or simply “antibody portion,” or “fragment”), as used herein, may be defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

An antibody applicable as an IL-1 inhibitor for the purpose of the invention specifically recognizes and binds IL-1α, IL-1β or IL-1R1 or any molecule participating in signal transduction pathway/s mediated by IL-1. It should be therefore noted that the term “binding specificity”, “specifically binds to an antigen”, “specifically immuno-reactive with”, “specifically directed against” or “specifically recognizes”, when referring to an epitope, specifically, a recognized epitope within the IL-1α, IL-1β or IL-1R1 or any molecule participating in signal transduction pathway/s mediated by IL-1 molecule, refers to a binding reaction which is determinative of the presence of the epitope in a heterogeneous population of proteins and other biologics.

More specific embodiments of the invention relate to an anti-IL-1α antibody and uses thereof for the methods of the invention.

In yet further embodiments, a protein-based IL-1 inhibitor may be any peptide or recombinant protein (including fusion proteins) that specifically blocks, disturbs and eliminates binding of IL-1 (specifically, IL-1α) to its cognate type 1 receptor, or otherwise block IL-1 mediated signaling.

As indicated above, in other embodiments, a protein-based IL-1 inhibitor, for example, an antibody, a polypeptide or a peptides used by the methods of the invention, can be prepared using recombinant DNA technology methods wherein an expression vector comprises nucleic acid sequence encoding the same, wherein the nucleic acid sequence is operably linked to a promoter. The expression vector can be delivered to, for example but not limited to, by methods of transformation, transfection, etc., a suitable host cell that allows expression of the peptide. Host cells comprising the expression vector are cultured under appropriate conditions and the peptide is expressed. In one embodiment the host cell is a mammalian cell, including human cell. In another embodiment, the host cell is bacterial, fungal or insect cell. In one embodiment the peptide is recovered from the culture wherein the recovery may include a step that leads to the purification of the peptide.

In yet further embodiments, the IL-1 inhibitor of the invention may be a small molecule, which is a low molecular weight organic compound that is not a polymer. The term “small molecule”, especially within the field of pharmacology, is usually restricted to a molecule that also binds with high affinity to a biopolymer such as protein, nucleic acid, or polysaccharide and in addition alters the activity or function of the biopolymer. The upper molecular weight limit for a small molecule is approximately 800 Daltons which allows for the possibility to rapidly diffuse across cell membranes so that they can reach intracellular sites of action. These compounds can be natural (such as secondary metabolites) or artificial. Non-limiting examples of small molecules include: Alkaloids, Glycosides, Lipids, Flavonoids, Nonribosomal peptides such as actinomycin-D, Phenazines, Phenols, Polyketide, Terpenes, including steroids and Tetrapyrroles.

It should be appreciated that the nucleic acid agents and the polypeptides used by the invention may be provided as isolated and purified molecules. As used herein, “isolated” or “substantially purified”, in the context of a polypeptide or nucleic acid molecule, means the polypeptide or nucleic acid has been removed from its natural milieu or has been altered from its natural state. As such “isolated” does not necessarily reflect the extent to which the polypeptide or nucleic acid molecule has been purified. However, it will be understood that a polypeptide or nucleic acid molecule that has been purified to some degree is “isolated”. If the polypeptide or nucleic acid molecule does not exist in a natural milieu, i.e. it does not exist in nature, the molecule is “isolated” regardless of where it is present.

Furthermore, the term “isolated” or “substantially purified”, when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

Still further, it must be appreciated that any known IL-1 inhibitor or blocker, specifically the inhibitors disclosed herein before or after, may be used by the methods of the invention. More specifically, any recombinant protein, peptide, antibody, nucleic acid or small molecule displaying IL-1 blocking activity, may be applicable for the purpose of the invention.

One of the most notable examples for recombinant gene products that are capable of inhibiting the IL-1 signaling is Kineret® (by Amgen, now Swedish Orphan BioVitrum), a recombinant non-glycosylated version of the human Anakinra (IL-1Ra) prepared in genetically modified Escherichia coli, which has been approved by the FDA for the treatment of rheumatoid arthritis. Another more advanced example is a chimeric molecule of the natural IL-1Ra with IL-1β (EPI-005) developed by Eleven Biotherapeutics that now undergoes initial clinical trials for topical application in refractory dry eye syndrome. It is noteworthy that the chimeric IL-1Ra occupies IL-1R with a greater affinity and duration than Anakinra.

Thus, according to certain specific embodiments, the IL-1 inhibitor used by the invention may be a recombinant IL-1Ra, specifically, Anakinra. In more particular embodiments, the IL-1 inhibitor used by the invention may be Kineret®.

Further, it is conceived that the present invention may apply various antibodies, polyclonal, monoclonal, chimeric, recombinant antibodies or antibody fragments that are capable of interfering with the IL-1 mediated signaling. Of particular relevance is Rilonacept also known as IL-1 Trap (Arcalyst® by Regeneron Pharmaceuticals), a soluble form of IL-1R decoy, a dimeric fusion protein consisting of ligand-binding domains of the extracellular portions of the human IL-1R1 and the IL-1RAcP accessory protein, linked in-line to the fragment-crystallizable portion (Fc region) of human IgG that binds and neutralizes IL-1α and IL-1β. Rilonacept was given an “Orphan Drug” status by the FDA and is used for the treatment of cryopyrin-associated periodic syndromes (CAPS), a spectrum of auto-inflammatory disorders. Another example is Canakinumab (Ilaris® by Novartis, previously known as ACZ885) is a human monoclonal antibody targeted at IL-1β. Canakinumab has been approved for the treatment of CAPS by the FDA and the European Medicines Agency.

Thus, in certain embodiments, the IL-1 inhibitor used by the invention may be of IL-1R decoy, more specifically, Rilonacept (Arcalyst®). In yet other embodiments, the IL-1 inhibitor of the invention may be a specific antibody directed against IL-1β. A specific embodiment of the invention relates to the use of Canakinumab (Ilaris®).

A number of other preparations of antibodies targeting IL-1 or IL1R1 have been more recently developed and are now under clinical trials. As IL-1α precursor is known to be released from infiltrating neutorphils and macrophages that are present in may clinical conditions, a neutralizing monoclonal IL-1α antibody, MABp1, has been developed by Xbiotech and is currently being tested in type 2 diabetes, advanced cancer, cancer cachexia, leukaemia, severe psoriasis, occlusive vascular disease and scarring acne vulgaris. Thus, according to certain specific embodiments, the IL-1 inhibitor used by the invention may be an IL-1α antibody, specifically, MABp1.

Further, an IL-1 receptor antibody, AMGI08 (licensed to AstraZeneca and MedImmune and now termed MEDI-78998), was tested in patients with rheumatoid arthritis and exhibited excellent safety but was only marginally more effective in relieving symptoms than Anakinra. AMGI08, when administered parenterally and not intra-articularly, has shown efficacy in the treatment of osteoarthritis. Thus, according to other specific embodiments, the IL-1 inhibitor used by the invention may be an antibody directed against IL-1R, for example, MG 108 (MEDI-78998).

Further, there is an accumulating experience with the production and clinical implementation of IL-1β neutralizing antibodies, owing to their specificity and long half-life. Neutralization of IL-1β is now emerging as an optimal strategy for some chronic diseases and being tested in several trials. Canakinumab, which has been mentioned herein above, for the treatment of CAPS, is currently in trials for type 1 diabetes and chronic obstructive pulmonary disease. The CANTOS study, the largest trial using an anti-cytokine drug, is testing whether neutralizing IL-1β will reduce cardiovascular events in high-risk patients. In addition to canakinumab, other anti-IL-1β antibodies, i.e. gevokizumab and LY2189102, are also in multiple clinical trials. Thus, according to certain specific embodiments, the IL-1 inhibitor used by the invention may be IL-1β neutralizing antibodies.

Therapeutic vaccines that induce the production of endogenous antibodies targeting epitopes on specific endogenous factors along the IL-1 signaling pathway have demonstrated efficacy and safety in humans in several settings. CYT-013 is a novel vaccine targeting IL-1β, for which a Phase I clinical trial has been initiated in patients with type 2 diabetes. It should be appreciated that in certain embodiments, the invention encompasses the use of the above-therapeutic vaccines as IL-1 inhibitors.

Orally active small molecules that target the release of active IL-1β have also been developed, including two caspase 1 inhibitors: pralnacasa (VX-740) and VX-765 (by Vertex). As pralnacasan was effective in vitro and in several animal models, the drug was tested in a clinical trial in rheumatoid arthritis. VX-765 has been administered to patients with Muckle-Wells syndrome leading to 40-70% decrease in markers of inflammation as well as a significant reduction in recurrent fevers and arthritis. As IL-1 blockade have been shown to prevent seizures in animal models, VX-765 is now being tested in patients with treatment resistant partial epilepsy, with apparent success.

In certain embodiments, the IL-1 inhibitor used by the invention may be caspase-1 inhibitors, for example, VX-740 and VX-765.

It has been known that the activation of caspase-1 with subsequent release of IL-1β requires a reduction in the levels of potassium in the cell. This process is controlled by the ATP receptor P2X purinergic receptor 7 (P2X7) and therefore small-molecule inhibitors of P2X7 have been developed and tested in humans. In patients with rheumatoid arthritis, the P2X7 inhibitor, AZD9056, resulted in a significant clinical improvement in joint inflammation. Other P2X7 inhibitors, CE-2245354 and GSK1482169, are currently being tested in Phase I and Ha trials.

The present invention contemplates an alternative clinical use of IL-1 inhibitors that is fundamentally distinct form the above mentioned clinical applications, namely for the purpose of preservation and retention of the ovarian follicular reserve in mammals, including humans.

In some embodiments of the invention, other drugs with similar activities, similar characteristics, similar biological properties, similar functionalities to interfere with the inflammation pathway, similar functionalities to interfere with the Interleukin-1 pathways, similar functionalities to block interleukin receptors, similar functionalities to otherwise affect a patients response to FSH and/or similar functionalities to improve a patients response to controlled ovarian hyperstimulation, are also contemplated by the invention.

It should be further appreciated that the invention encompasses the use of any combination of at least two of the IL-1 inhibitors mentioned herein before, or any combination thereof with any additional therapeutic agent.

As shown by the invention, depletion of IL-1α results in an increased expression of FHSR on ovarian granulosa cells thereby enhancing responsiveness of the cells to gonadotropin treatment.

Thus, in yet another aspect, the invention provides an IL-1 inhibitor as defined by the invention, for use in a method of improving response of the treated mammalian subject to a controlled ovarian hyper stimulation (COH).

More specifically, in certain embodiments, the IL-1 inhibition provided by the invention may improve, elevate, increase, extend, expand or enlarge a response of a mammalian subject to COH treatment. Such elevation, improvement or increase may be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.Moreover, with regards to the above, it is to be understood that, where provided, percentage values such as, for example, 10%, 50%, 100%, end even more, etc., are interchangeable with “fold change” values, i.e., 0.1, 0.5, 1.2, 5, etc.

In some specific embodiments, improving response to COH may be specifically applicable in case of a subject diagnosed or classified as a poor ovarian responder (POR).

It should be noted that the term “response”, “responsiveness”, “responsive” or “responder” to treatment with a specific therapeutic agent, for example, gonadotropins, refers to an improvement in at least one relevant clinical parameter as compared to an untreated subject diagnosed with the same pathology (e.g., the same type, stage, degree and/or classification of the infertility associated condition), or as compared to the clinical parameters of the same subject prior to said treatment.

The term “non-responder” or “non-responsive” to treatment using a specific therapeutic agent, refers to a patient not experiencing an improvement in at least one of the clinical parameter and is diagnosed with the same condition as an untreated subject diagnosed with the same pathology (e.g., the same type, stage, degree and/or classification of the infertility condition), or experiencing the clinical parameters of the same subject prior to such treatment.

More specifically, POR to ovarian stimulation usually indicates a reduction in follicular response, resulting in a reduced number of retrieved oocytes. To define the poor response in IVF, at least two of the following three features must be present: (i) advanced maternal age or any other risk factor for POR; (ii) a previous POR; and (iii) an abnormal ovarian reserve test (ORT). Two episodes of POR after maximal stimulation are sufficient to define a patient as poor responder in the absence of advanced maternal age or abnormal ORT. By definition, the term POR refers to the ovarian response, and therefore, one stimulated cycle is considered essential for the diagnosis of POR. However, patients of advanced age with an abnormal ORT may be classified as poor responders since both advanced age and an abnormal ORT may indicate reduced ovarian reserve and act as a surrogate of ovarian stimulation cycle outcome. As mentioned above, in some embodiments, the invention is directed to improve response of a subject diagnosed or classified as a POR to COH Treatment.

In further specific embodiments, the method of the invention improves response of the treated subject to COH that may comprise administration of at least one gonadotropin. Under gonadotropins is meant glycoprotein hormones secreted by gonadotropin producing cells (that are basophilic cell of the anterior pituitary specialized to secrete follicle-stimulating hormone and luteinizing hormone), including the follicle-stimulating hormone (FSH) and luteinizing hormone (LH) secreted by the anterior pituitary gland, and the placental chorionic gonadotropins (hCG) secreted by the placenta. The gonadotropin-releasing hormone family also includes the gonadotropin-releasing hormone (GnRH), also known as luteinizing-hormone-releasing hormone (LHRH) and luliberin, is a trophic peptide hormone responsible for the release of FSH and LH from the anterior pituitary. GnRH is synthesized and released from GnRH neurons within the hypothalamus, thus constituting the initial step in the hypothalamic-pituitary-gonadal axis.

Thus, in certain embodiments, the invention provides IL-1 inhibitors for use in a method for improving response of a subject to any treatment regimen comprising at least one of FSH, GnRH, gonatotrophin releasing factor (GRF) either alone or in combination with FSH, human chorionic gonadotropin (hCG), or an analogue thereof.

It should be appreciated that in certain embodiments, the IL-1 inhibitor compound/s used by the invention or any formulation or composition thereof may be adapted for use before, simultaneously with, after or any combination thereof with said at least one gonadotropin.

In certain embodiments, the treated subject is undergoing an in vitro fertilization (IVF) treatment. Therefore, some embodiments of the invention provide the use of IL-1 inhibitor/s for improving response to COH of subjects that undergoing an IVF treatment.

In yet a further aspect, the invention provides an IL-1 inhibitor for use in a method of treating, inhibiting, preventing or ameliorating infertility or related conditions in a mammalian subject.

Further in this connection, under infertility is meant a woman who is unable to conceive as well as being unable to carry a pregnancy to full term. Demographically infertility is defined as childlessness in a population of women of reproductive age, whereas epidemiologically infertility refers to “trying for” or “time to” a pregnancy, generally in a population of women exposed to a probability of conception. Infertility rates have increased by 4% since the 1980s, mostly from problems with fecundity due to an increase in age.

According to the WHO infertility is defined by the failure to achieve a clinical pregnancy after 12 months or more of regular unprotected sexual intercourse in absence of known reproductive pathology or breastfeeding or postpartum amenorrhea. It should be appreciated that the invention relates to the treatment of both, primary and secondary infertility. Primary infertility is infertility in a couple who have never had a child. Secondary infertility is failure to conceive following a previous pregnancy. Researchers commonly base demographic studies on infertility prevalence on a five-year period. Practical measurement problems, however, exist for any definition, because it is difficult to measure continuous exposure to the risk of pregnancy over a period of years. Among many biological and other causes of infertility in women, of particularly relevance here is an advanced maternal age, which, among others, is strongly associated with a reduced number of follicles. Other important causes for infertility are inflammatory conditions such as endometriosis and Crohn's disease that are also associated with reduced fertility. Therefore, in certain and non-limiting embodiments, the invention may be applicable for treating any infertility, conditions associated with inflammation.

Particularly relevant to the present invention clinical conditions associated with female infertility, primarily premature ovarian failure (POF), also known as premature ovarian insufficiency (POI), primary ovarian insufficiency, premature menopause, hypergonadotropic hypogonadism, is the loss of function of the ovaries before age 40. A commonly cited triad for the diagnosis of POF is amenorrhea, hypergonadotropinism, and hypoestrogenism. Genetic causes include gonadal dysgenesis characterized by a progressive loss of follicles in the developing fetal gonads leading to extremely hypoplastic and dysfunctioning gonads in adults, mainly composed of fibrous tissue (streak gonads, i.e. replacement with a functionless tissue). The accompanying hormonal failure also prevents the development of secondary sex characteristics, resulting in a sexually infantile female appearance and infertility.

Still further, in order to appreciate applicability of the present invention, it should be understood that the normal and abnormal folliculogenesis is strongly linked to the levels of circulating gonadotropins and female sex hormones responsible for sexual maturation and reproductive function in mammals. As mentioned above, under gonadotropins is meant the major hormones secreted by gonadotropin producing cells, including FSH, LH and hCG. Under female sex hormones is meant the major sex steroid hormones produced by the ovaries, including estrogen and progesterone (the ovaries also produce low levels of the male hormone, testosterone).

Further, the present invention is applicable while considering the cyclical nature of release of female gonadopropins and sex hormones that determine the ovarian cycle (changes that occur in the ovarian follicles) and the uterine cycle (changes in the endometrial lining of the uterus). Under the ovarian cycle is meant the follicular phase, ovulation, and the luteal phase, whereas under the uterine cycle—the menstruation, proliferative phase, and secretory phase. In this context, the ovarian cycles also refer to the characteristic levels of the gonadotropin and sex hormones and the regulatory feedback between them. More specifically, the ovarian cycle is characterized with a rise in FSH levels at the follicular phase, the LH and FSH surge prior to ovulation, rise in estrogen levels before ovulation and subsequent estrogen drop, and further a rise in progesterone levels produced by the corpus luteum at the luteal phase. The uterine cycle responds to the hormonal changes produced by the pituitary and ovaries, whereby estrogen induces formation of a new layer of endometrium in the uterus during the proliferative phase and progesterone plays a role during the secretory phase in making the endometrium receptive to implantation of the blastocyst by increasing blood flow and uterine secretions and reducing contractility of the smooth muscle. If fertilization occurs, corpus luteum is retained and produces progesterone to maintain the new pregnancy, the syncytiotrophoblast (also the outer layer of the placenta) produces human chorionic gonadotropin (hCG), which is tested by most pregnancy tests. In absence of fertilization or pregnancy, menstruation occurs; eumenorrhea denotes a normal menstruation (3-5 days).

Thus in certain embodiments, the present invention is particularly applicable alleviation of female infertility resulting from hormonal abnormalities, such as hypogonadism. Under hypogonadism (also hypoestrogenism in females) is meant a diminished functional activity of the gonads, i.e. ovaries, resulting in diminished sex hormone biosynthesis. Apart from estrogen, other hormones produced by the gonads which may be decreased by hypogonadism include progesterone, DHEA and anti-Müllerian hormone. One of the apparent outcomes of hypoestrogenism is a reduced ovulation, which depending on the degree of severity, may result in partial or complete infertility.

It should be appreciated that in certain embodiments, the invention is applicable for any condition or disorder associated with reproduction, fertility or infertility. The term “reproduction” as used herein, refers to the biological process by which new offspring individual organisms are produced from their parents. The present invention relates to sexual reproduction, which typically is generally defined as the creation of a new organism by combining the genetic material of two organisms.

In more specific embodiments, infertility related conditions may include but are not limited to incomplete follicle development, hypogonadism, reduced ovulation, premature ovarian failure, polycystic ovarian syndrome, endometriosis and sterility.

In some specific embodiments, the methods and uses of the invention may be applicable for the treatment of PCOS. Under polycystic ovary syndrome (PCOS, also called hyperandrogenic anovulation (HA) or Stein-Leventhal syndrome) is meant an endocrine system disorder among women of reproductive age diagnosed as enlarged ovaries that contain small collections of fluid (cysts) that become apparent during an ultrasound exam. It has been previously shown that PCOS patients have an increased prevalence of insulin resistance (IR) and related disorders and excess androgenic hormones that reflect low-grade chronic inflammation. The most common immediate symptoms are anovulation that results in irregular menstruation, amenorrhea, and ovulation-related infertility.

Endometriosis, a chronic inflammatory condition is associated with infertility and lower fertilization and pregnancy rates compared to unaffected IVF patients. Elevated levels of TNFα in the follicular fluid were correlated with poor oocytes quality while TNFα blockage improves IVF outcomes in young infertile women. Thus, further embodiments of the invention relate to the applicability of methods and uses of IL-1 inhibitors for treating endometriosis and/or other inflammatory conditions.

More specifically, endometriosis is a pathological condition characterized by ectopic endometrial implants, usually in the peritoneal cavity. Active endometriosis is characterized by hypervascularization both within, and around the implant. Endometriosis is manifested during the reproductive years; it has been estimated that endometriosis occurs in roughly 5-18% of women. Some symptoms may develop at the site of active endometriosis; the main, but not universal symptom is pelvic pain at various manifestations. Other symptoms are: painful sexual intercourse (dyspareunia) or cramping during intercourse, as well as pain during bowel movements and/or urination. Endometriosis is common in women with infertility problems. Endometriosis lesions react to hormonal stimulation by proliferation and angiogenesis. There is a high VEGF level in the peritoneal fluid of patients with endometriosis and its production is stimulated by both Estrogen and Progesterone.

In some examples, women who are known to be poor and/or low responders may be refused access to IVF clinics outright, ostensibly so as to not skew the clinic's success rate. In other examples, women who are known to be poor or low responders may have different clinical protocols applied to overcome their poor response.

Patients who consistently fail to respond to FSH and repeated ovarian stimulation may end up with ovarian failure. Ovarian failure may result in infertility, due to among other factors, the failure to produce sufficient amounts of estrogen in some instances, infertility is irreversible. Poor and/or low responses to Gonadotropins (e.g., FSH) e.g., not developing follicles for ovulation after one cycle of IVF treatment, in general are not necessarily age related; young patients may also experience low and/or poor response. In some examples, ovarian reserve testing can predict poor responses to gonadotropins. Other functional tests can also be used to predict poor responses.

Still further, it must be understood that the IL-1 inhibitor/s used by the invention may be applicable in the treatments and prevention of infertility or of any condition associated or linked to infertility, reproduction or fertility. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

More specifically, as used herein, “disease”, “disorder”, “condition”, “pathology” and the like, as they relate to a subject's health, are used interchangeably and have meanings ascribed to each and all of such terms.

In certain embodiments, the IL-1 inhibitor/s used by the invention, are particularly applicable for subject/s treated with COH medications. More specifically the COH medications comprise at least one gonadotropin. In certain embodiments, gonadotropins include but are not limited to FSH, GnRH, GRF either alone or in combination with FSH, hCG, or an analogue thereof.

In more specific embodiments, such subject may be a subject diagnosed or classified as a poor ovarian responder (POR).

In certain embodiments, the IL-1 inhibitor compound or any formulation or composition thereof may be adapted for use before, simultaneously with, after or any combination thereof with at least one gonadotropin of the COH treatment.

Thus, the invention provides IL-1 inhibitor/s or blocker/s that may be used either before gonadotropin treatment, thereby sensitizing the patient to gonadotropin treatment, or after the gonadotropin treatment. It should be noted that in some embodiments, the IL-1 inhibitors may be used simultaneously with said gonadotropins, to enhance the effect of said treatment, thereby providing a combined therapy that will be discussed herein after.

Inflammation may adversely affect both IVF outcomes and the ovarian reserve. Interleukin (IL)-1 functionality and activity may offer a new insight into the mechanisms responsible for oocytes loss as well as practical intervention such as IL-1 blockade that aims to slow down the rate at which oocytes are eliminated on one hand and improve IVF outcomes.

Thus, in more specific embodiments, the uses of the IL-1 according to the invention may be applicable for treating a subject undergoing an IVF treatment.

When referring to IVF is meant a method for assisted reproductive technology in a female that involves monitoring and stimulating an ovulatory process (ovarian hyperstimulation), removing ovum or ova from the ovaries and letting sperm fertilize them in a fluid medium in a laboratory. Specifically in this context, ovarian hyperstimulation is the stimulation to induce development of multiple follicles, a response to which is predicted on the basis of a number of factors, among others, the antral follicle count and levels of AMH. The resulting prediction of e.g. poor or hyper-responders determines the protocol and dosage for ovarian hyperstimulation. Ovarian hyperstimulation also includes suppression of spontaneous ovulation using a GnRH agonist (long) protocol or GnRH (short) antagonist protocol. In a standard long GnRH agonist protocol the day when hyperstimulation treatment is started and the expected day of later oocyte retrieval can be chosen to conform to personal choice, while in a GnRH antagonist protocol it must be adapted to the spontaneous onset of the previous menstruation.

As mentioned above, the present invention enables to achieve productive advantage and increased pregnancy rates at a middle and advanced reproductive age, as demonstrated by the invention in Examples I and II. Thus in certain embodiments, the present invention particularly applies to females in an advanced reproductive age that have already reduced number of follicles, as well as females suffering from infertility and other clinical conditions associated with a reduction in the number of follicles. In this connection, for average women population of follicles at puberty is estimated at 180,000 (range from 25,000 to 1.5 million). By virtue of the “inefficient” nature of folliculogenesis only about 400 of these follicles will ever reach ovulation. At early menopause, only 1,000 follicles remain. It seems likely that early menopause occurs for women with low follicular population at birth and late menopause occurs for women with high population at birth.

Thus, it should be noted that the IL-1 inhibitor/s of the invention, by blocking or inhibiting the IL-1 activity, extend the fertility period of the treated subject, and/or prolong the duration of fertility and/or postpone the menopause. In some embodiments the subject may be over 25, 28, 29, 30, 32, 33, 34, 35, 36, 37, 38, 39, 40, 41, 42, 43, 44, 45, 46, 47, 48, 49 and 50 years old. As such, certain embodiments of the invention provide IL-1 inhibitors that may be used for prolonging ovarian lifespan of the subject.

Overall, in certain embodiments, the IL-1 inhibitors provided by the invention are applicable in improving the quantity as well as the quality of ovarian follicles, and more specifically, improving the quality and quantity of oocytes. By improving quality of oocytes is meant improving the maturation of oocytes, and/or improving the synchrony of nuclear, cytoplasmic and/or membranous oocyte maturation, and/or improving the fertility of oocytes, and/or improving the rate of implantation of oocytes by in vitro maturation and fertilization.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

As indicated above, the IL-1 inhibitor/s provided by the present invention may be used for the treatment of a “pathological disorder”, which refers to a condition, in which there is a disturbance of normal functioning, any abnormal condition of the body or mind that causes discomfort, dysfunction, or distress to the person affected or those in contact with that person. As used herein, “disease”, “disorder”, “condition” and the like, as they relate to a subject's health, are used interchangeably and have meanings ascribed to each and all of such terms.

By “patient” or “subject in need” it is meant any organism who may be affected by the above-mentioned conditions, and to whom the therapeutic methods herein described is desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the IL-1 inhibitors or any formulations or compositions thereof, to the patient includes both self-administration and administration to the patient by another person.

The term “treatment or prevention” as used herein, refers to the complete range of therapeutically positive effects of administrating to a subject including inhibition, reduction of, alleviation of, and relief from, infertility or related condition/s and illness, symptoms or undesired side effects or related disorders. More specifically, treatment or prevention of occurrence or re recurrence of the disease in response to a treatment with a non-effective, or deleterious therapeutic agent, includes the prevention or postponement of development of the condition, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

More specifically, for prophylactic applications, the IL-1 inhibitor or any formulations or compositions thereof, may include a prophylactic effective amount of the active ingredient. The term “prophylactically effective amount” is intended to mean that amount of a pharmaceutical composition that will prevent or reduce the risk of occurrence or recurrence of the biological or medical event that is sought to be prevented in a tissue, a system, animal or human by a researcher, veterinarian, medical doctor or other clinician. In prophylactic applications, the compositions of the invention are administered to a patient who is at risk of developing the disease state to enhance the patient's resistance. Such an amount is defined to be a “prophylactically effective dose”. In this use, the precise amounts again depend upon the patient's state of health and general level of immunity, but generally range from 0.001 to 1000 mg per dose.

Still further, the invention provides IL-1 inhibitor/s as defined herein above for use in a method for sensitizing and increasing the responsiveness of follicular cells to gonadotropin/s.

Increase of the in vivo responsiveness and sensitivity of follicular cells of any developmental stage to gonadotropin/s, in a subject in need thereof, thereby reducing the required effective amount of gonadotropin/s, that are required for achieving the desired therapeutic effect. In some embodiments, the increased sensitivity is a result of an increased expression of FSHR in said follicular cells.

In yet further embodiments, the invention provides at least one IL-1 inhibitor for use in a method for ex-vivo or in vitro sensitizing follicular cells to at least one gonadotropin/s.

In yet a further aspect, the invention provides the use of the IL-1 inhibitors of the invention for preserving ovarian tissue, whether in vivo in a subject in need thereof, or ex vivo. More specifically, in certain embodiments, the invention provides methods for in vivo or in vitro, specifically, ex-vivo preservation of ovarian follicular reserve in a subject exposed to or that is expected to be exposed to genotoxic compound/s or procedures that may damage the ovarian reserve. In certain embodiments, such genotoxic procedures or compounds include chemotherapeutic treatment. A chemotherapeutic drug may be used alone or in combination with another chemotherapeutic drug or with other forms of cancer therapy, such as a biological drug, radiation therapy or surgery.

In some embodiments, the methods of the invention may in vivo preserve, in case performed prior to exposure to a chemotherapeutic agent, the ovarian follicular reserve. “Chemotherapeutic agent” or “chemotherapeutic drug” (also termed chemotherapy) as used herein refers to a drug treatment intended for eliminating or destructing (killing) cancer cells. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, hair follicles and ovarian follicles.

Chemotherapeutic drugs affect cell division or DNA synthesis and function and can be generally classified into groups, based on their structure or biological function. The present invention generally pertains to chemotherapeutic agents that are classified as alkylating agents, anti-metabolites, anthracyclines, plant alkaloids, topoisomerase inhibitors, and other anti-tumor agents.

In yet other embodiments, the IL-1 inhibitors of the invention may be used for ex-vivo maintaining the ovarian follicle reserve in an ovarian tissue obtained from a subject expected to be exposed to said genotoxic treatment.

As mentioned herein before, the IL-1 inhibitors or any formulations or compositions provided by the invention optionally further comprise at least one pharmaceutically acceptable excipient or carrier. As used herein “pharmaceutically acceptable carrier” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

The pharmaceutical composition of the invention can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration,” “administered systemically,” “peripheral administration” and “administered peripherally” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Regardless of the route of administration selected, the IL-1 inhibitor compounds of the present invention, which may be used in a suitable hydrated form, and/or the pharmaceutical compositions of the present invention, are formulated into pharmaceutically-acceptable dosage forms by conventional methods known to those of skill in the art.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

Pharmaceutical compositions used to treat subjects in need thereof according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Local administration to the area in need of treatment may be achieved by, for example, local infusion during surgery, topical application, directs injection into the specific organ, etc.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

In particular embodiments, the unit dosage formulations are those containing a daily dose or sub-dose, as herein above recited, or an appropriate fraction thereof, of an active ingredient.

The IL-1 inhibitors or blockers used by the invention, or any formulations or compositions thereof, if delivered in a solid form, may be prepared in any suitable form such as pellets, discs, rods or microspheres. These may be administered to the subject either by implantation of a composition unit (in the form of a pellet, disc or rod) or by injection, intramuscular, subcutaneous or intraperitoneal (in the form of a suspension of mini-rods or micro-spheres). Alternatively, a composition in a solid form may be administered by feed as an oral composition.

In case of implantable composition, the size of such composition in accordance with the present invention will be determined both by the size of the organ in which implantation thereof is intended, i.e. it should not be too big, and by practical limitations, i.e. the implantable composition should not be too small so as to render it difficult for manipulation. Thus, for example, a disc having a diameter of about 1 to 10 mm and a thickness of about 0.01 to 2 mm has been found in the art to be suitable for implantation.

It should be noted that the invention further encompass the use of sustained release compositions. “Sustained release” is understood to mean a gradual release of active compound in a controlled manner. Such sustained release formulations of active compounds may be solid and may be prepared in any suitable form such as pellets, discs or rods, or encapsulated in microspheres. Active compounds may be also administered by methods including implementation of a unit of active compound in any suitable form, such as long lasting implants.

A further aspect of the invention relates to an ex-vivo method of improving oocyte quality and/or survival. More specifically, the method of the invention may comprise an ex-vivo contacting an oocyte in a cell culture with an effective amount of at least one IL-1 inhibitor or any composition or formulation thereof that blocks or inhibits IL-1 activity, thereby improving oocyte quality and/or survival.

In certain embodiments, the oocyte may be maintained under conditions for in-vitro fertilization (IVF).

In further specific embodiments, the oocyte may be comprised in an ovary or an ovarian tissue.

Still further, the invention provides a cell culture comprising an oocyte and an exogenously added effective amount of at least one IL-1 inhibitor or any composition or formulation thereof. It should be noted that the ex vivo methods of the invention for improving oocyte quality and survival are intended for use in a human subject, however, it must be appreciated that the methods may be applicable for any mammalian subject as disclosed by the invention. In certain embodiments, the ex vivo method of the invention may be applicable for treating domestic and non-domestic mammals such as canine and feline subjects, bovine, simian, equine and rodents, specifically, murine subjects. In more specific embodiments, the ex vivo methods of the invention may be applicable for improving the quality of murine oocytes in a cell culture.

By improving quality of oocytes is meant improving the maturation of oocytes, and/or improving the synchrony of nuclear, cytoplasmic and/or membranous oocyte maturation, and/or improving the fertility of oocytes, and/or improving the rate of implantation of oocytes by in vitro maturation and fertilization.

It should be appreciated that the invention further provides the use of at least one IL-1 inhibitor in the preparation of a composition for retaining ovarian follicle reserve in a mammalian subject, treating infertility and improving responsiveness to COH.

A further aspect of the invention relates to a method for retaining ovarian follicle reserve in a mammalian subject by blocking, inhibiting or reducing the activity of IL-1 in the treated subject. In more specific embodiments, the method of the invention may comprise the step of administering to the subject an effective amount of at least one IL-1 inhibitor or any composition or formulation thereof.

In some embodiments, blocking or inhibition of IL-1 activity in the treated subject may lead to reduction in ovarian follicular atresia, thereby retaining ovarian follicle reserve in the subject.

In more specific embodiments, reduction in ovarian follicle atresia may be a result of at least one of, increase in the expression or activity of pro-survival proteins, decrease in the expression or activity of pro-apoptotic proteins and decrease in the expression or activity of pro-inflammatory cytokines, in ovarian cells of the treated subject. In some non-limiting examples, such ovarian cells may be cells comprised within an ovarian follicle. In some other embodiments, such ovarian cells may be ovarian-stroma cells.

In further embodiments, the blocking or inhibition of IL-1 caused by the method of the invention may further result in increased expression of FSHR in cells comprised within ovarian follicles of said subject. In certain and specific non-limiting embodiments depletion of IL-1α, by the method of the invention may result in increased expression of FSHR in follicular granulosa cells.

In some embodiments, an increased expression of FSHR in said follicular cells increases, enhances and promotes ovarian follicular recruitment and maturation.

In some specific embodiments, the IL-1 inhibitor/s used by the methods of the invention block/s, inhibit/s or reduce/s the activity, expression, stability, post-transcriptional or posttranslational processing of at least one of IL-1α, IL-1β, Interleukin-1 receptor type 1 (IL-1R1) or of any molecule participating in signal transduction pathways mediated by IL-1.

More specifically, IL-1 inhibitor/s applicable for the method of the invention may be any synthetic or natural protein compound, peptidomimetic compound, nucleic acid compound, a small molecule or any combinations thereof.

In further aspects, the method of the invention may be applicable for improving response of the mammalian subject to a COH.

In more specific embodiments, such subject may be diagnosed or classified as a POR.

More specifically, the COH may comprise administration of at least one gonadotropin.

In certain embodiments, the IL-1 inhibitor compound or any formulation or composition thereof used by the method of the invention, may be adapted for use before, simultaneously with, after or any combination thereof with said at least one gonadotropin.

In more specific embodiments, the method of the invention may be applicable for subject/s undergoing an in vitro fertilization (IVF) treatment and\or insemination (IUI) with or without gonadotropin stimulation.

In a further aspect, the methods of the invention are particularly applicable for treating, inhibiting, preventing or ameliorating infertility or infertility-related conditions in a mammalian subject. More specifically, the method of the invention may comprise the step of administering to the subject an effective amount of at least one IL-1 inhibitor or any composition or formulation thereof.

In some embodiments, the method of the invention may be applicable for subject/s treated with COH medications.

In more specific embodiments, such subject may be diagnosed or classified as a POR.

Still further, more particular embodiments relate to the methods of the invention wherein the treated subject/s are also treated with COH medications that comprise at least one gonadotropin.

In more specific embodiments, the IL-1 inhibitor compound used by the methods of the invention, or any formulation or composition thereof may be adapted for use before, simultaneously with, after or any combination thereof with said at least one gonadotropin.

In more specific embodiments, the methods of the invention are applicable for subject/s undergoing an in vitro fertilization (IVF) treatment.

Still further, the invention further provides methods for sensitizing and increasing responsiveness of ovarian follicular cells to gonadotropin/s treatment. More specifically, the method of the invention may comprise the step of administering to the subject an effective amount of at least one IL-1 inhibitor or any composition or formulation thereof.

In some embodiments of the invention, the therapy, i.e. the IL-1 inhibitors of the invention, may be provided via a pharmaceutical carrier at a therapeutically effective dose. In some embodiments of the invention, the therapy may be provided in a solid, semi-solid or liquid dosage type preparation. Preparations may include, for example, tablets, pills, powders, capsules, liquids, suspensions or other preparations.

In some embodiments of the invention, the therapy may be provided via a sustained and/or slow release carrier formulation such as semi-permeable polymer carriers in the form of suppositories or microcapsules.

In some embodiments of the invention, the therapy may be provided via an implant, such as an osmotic pump for slow and/or sustained release over an extended period of time. In some embodiments of the invention, the therapy may be provided via subcutaneous injection. In some embodiments of the invention, the therapy may include high specific formulations and/or other therapeutically effective concentrations. In some embodiments of the invention there may be a kit for providing an optimal therapeutic dose. Embodiments exemplifying such kit are disclosed herein after.

In some embodiments of the invention, the therapy configured for subcutaneous dosage may have an acceptable osmolality such that the injection does not significantly increase pain perception or a burning feeling after injection. In some embodiments of the invention, the therapy may be provided by other means.

The therapy may be provided in advance of an upcoming FSH dosage. The therapy may be provided a number of days prior to the FSH dosage, for example between 1 and 5 days prior to the FSH dosage, for example an FSH dosage within an IVF therapy regimen.

In some embodiments of the invention, the desired outcome from the therapy described herein and above would be enhancement of the FSH receptors on granulosa cells, resulting in a better response to treatment regimen, meaning, in some examples, higher oocyte yield per cycle.

In some embodiments of the invention, the therapy may include providing a formulation. In some embodiments of the invention IL-1 inhibitor/s therapy will be started prior to the FSH analog administration. In IVF patients, FSH analogs administration may start at day 3-5 of the cycle. The IL-1 inhibitor can be started earlier, for example at day one of a fertility cycle. Pharmaceutical formulations of the therapy may include one or more adjuvants, including for example, preservatives, wetting agents, emulsifying agents and dispersing agents.

Pharmaceutical formulation of the therapy may, in some embodiments of the invention, include between 1 and 1000 mM of a buffer and/or any other pharmaceutically acceptable excipient which stabilizes the pH of a pharmaceutical preparation such as one or more of: citrate-buffers, succinate-buffers, histidine-buffers, acetate-buffers and phosphate-buffers other amino acid-buffers.

In some embodiments of the invention, the buffer may be configured to maintain a pH, wherein the pH of the formulation may be from 5.5 to 7.5.

In some embodiments of the invention, the pharmaceutical formulation may also include a pharmaceutically acceptable excipient used to protect protein formulations against interfacial stresses like agitation and shearing such as a surfactant.

A therapy that includes a pharmaceutical formulation may include a bulking agent. Bulking agents may include mannitol, glycine, polyethylene glycol and sorbitol and/or other bulking agents.

In some embodiments of the invention, the pharmaceutical formulation may also include pharmaceutical acceptable excipients that act as stabilizer. These stabilizers may include sugars, amino acids, albumine, human serum albumin (HSA), bovine serum albumin (BSA), salts (sodium chloride, magnesium chloride, calcium chloride), chelators, polyols, cyclodextrine, and or poly-ethylenglycols (PEG) that protect the active pharmaceutical ingredient and/or the formulation from chemical and/or physical degradation during manufacturing, storage and application.

In some embodiments of the invention, the formulation may include agents configured to modulate the tonicity of the formulation. The formulation can be hypotonic, isotonic or hypertonic.

In some embodiments of the invention, the formulation may be diluted by a pharmaceutical acceptable excipient. A diluting agent may include saline, glucose, Ringer, bacteriostatic water for injection (BWFI) and aqueous buffer solutions.

The IL-1 inhibitor/s therapy according to the invention may be provided in a number of different possible doses. A patient may receive a dosage between 0.01 ng and 1000 mg of the formulation. For example, between 0.1 ng and 900 mg, or between 1.00 ng and 800 mg, or between 1.00 ng and 700 mg, or between 1.00 ng and 600 mg or between 1.00 ng and 500 mg, or between 1.00 ng and 400 mg, or between 1.00 ng and 300 mg, or between 1.00 ng and 200 mg, or between 1.00 ng and 100 mg, or between 10 ng and 100 mg. The therapy may be provided in conjunction with one or a plurality of other pharmaceuticals, pharmaceutical carriers or other additives.

As shown in Example VII, treatment with Anakinra resulted in improved fertilization rate and increased expression of FSHR. Thus, in some specific and non-limiting embodiments, when Anakinra is used as an IL-1 inhibitor, a daily dose of between about 0.1 mg/Kg-1000 mg/Kg of body weight, specifically, about 0.1, 0.2, 0.3, 0.4, 0.5, 0.6, 0.7, 0.8, 0.9, 1.0 mg/Kg to about 1000 mg/Kg, about 900, 800, 700, 600, 500, 400, 300, 200, 100/mg/kg. In yet more specific embodiments the an appropriate daily dose of between about 1.0 mg/Kg to about 100 mg/Kg, specifically, about 1, 2, 3, 4, 5, 6, 7, 8, 9, 10, 20, 30, 40, 50, 60, 70, 80, 90, 100 mg/kg may be administered. Still further specific embodiments relate to an effective daily amount that may be about 2 mg/Kg of body weight. In further embodiments for a patient ranging between 60-75 kg, a daily effective amount of about 100 to about 200 mg may be effective, specifically, 10, 110, 115, 120, 125, 130, 135, 140, 145, 150, 155, 160, 165, 170, 175, 180, 185, 190, 195, 200 mg, or more.

The formulation may be prepared for injection via a number of methods, including dialysis, ultrafiltration-diafiltration, addition and mixing, reconstitution and/or lyophilisation. The syringe may include additives such as a carrier or suspension, including, for example, water, ethanol, polyol, an isotonic buffered saline solution, lipophilic solvents or vehicles, such as fatty oils, synthetic fatty acid esters, triglycerides, or aqueous injection suspensions that comprise viscosity-increasing substances for example, sodium carboxymethylcellulose, sorbitol and/or dextran, and/or mixtures.

The injection may be subcutaneously. In some embodiments of the invention the injection may be intravenous, intramuscular, intraarterial, subcuticular, intraarticular, subcapsular, intrathecal, intracapsular, intraorbital, intracardiac, intradermal, intraperitoneal, transtracheal, subarachnoid, intraspinal, epidural and/or intrasternal.

In one embodiment, compositions of this invention, e.g., the therapy, are pharmaceutically acceptable. In one embodiment, the term “pharmaceutically acceptable” may in some embodiments of the invention, refers to any formulation which is safe, and provides the appropriate delivery for the desired route of administration of an effective amount of at least one compound for use in the present invention. This term, may in some embodiments of the invention, refer to the use of buffered formulations as well, wherein the pH is maintained at a value, the value, may for example range from pH 4.0 to pH 9.0, in accordance with the stability of the compounds and route of administration.

In one embodiment, an IL-1 inhibitor used in the methods of this invention may be administered alone or within a composition. In another embodiment, compositions comprising conjugates in admixture with conventional excipients, i.e. pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and/or polyvinyl pyrrolidione.

In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In general, the doses utilized for the above described purposes will vary, but will be in an effective amount to exert the desired effect. As used herein, the term “pharmaceutically effective amount” may in some embodiments of the invention refer to an amount of a compound of the therapy described hereinabove which will produce the desired alleviation in symptoms or other desired phenotype in a patient.

In some embodiments of the invention, the concentrations of the compounds will depend on various factors, including the nature of the condition to be treated, the condition of the patient, the route of administration and the individual tolerability of the compositions.

It will be appreciated that the actual amounts of active compound in a specific case will vary according to the specific compound being utilized, the particular compositions formulated, the mode of application, and the particular conditions and organism being treated. Dosages for a given host can be determined using conventional considerations, e.g., by customary comparison of the differential activities of the subject compounds and of a known agent, e.g., by means of an appropriate, conventional pharmacological protocol.

In one embodiment, the compounds of the invention may be administered acutely for acute treatment of temporary conditions, or may be administered chronically, especially in the case of progressive, recurrent, or degenerative condition. In one embodiment, one or more compounds of the invention may be administered simultaneously, or in another embodiment, they may be administered in a staggered fashion. In one embodiment, the staggered fashion may be dictated by the stage or phase of the condition being treated.

The therapy may be used, in some embodiments of the invention in preparation of a composition for the treatment of low ovarian reserve, and/or for improving controlled ovarian hyperstimulation in mammals.

In some embodiments of the invention, a subject, for example a woman who does not test as a poor responder, e.g., a putative responder, will nevertheless be provided therapy. In some examples, this therapy may be to benefit from an overall reduction of exogenous hormones.

The type, nature, duration, dosage and/or other characteristics of the formulation of the therapy may take into account body mass index (BMI) type of sub fertility, duration of sub fertility, external factors, stress levels, smoking, pelvic surgery, age of mother's menopause, and/or degree and extent of medical disorders such as hypertension or diabetes.

Therapy may include the co-implementation or partial co-implementation of other therapies for poor responders, including, for example, high stimulation protocols where the patient is provided with an extra-high dose of stimulation medications. In some embodiments of the invention, other protocols may include minimal stimulation protocols or alternative stimulation protocols, including for example, OCP/MicroFlare Lupron with high FSH dosing, High FSH/HMG dosing with GnRh antagonist, Estrogen and Antagonist start in 2nd part of luteal phase, combination MicroFlare and Antagonist with high FSH/HMG dosing Letrozole or Clomiphene and FSH/HMG, Estrogen/progesterone in previous cycle day 2. In some embodiments of the invention, other protocols may also include natural cycle IVF, acupuncture and other forms of alternative medicine, and synthetic estrogens such as oral contraceptives, femtrace, estrace, and/or gynodiol.

In some embodiments of the invention, the therapy may be provided to any patient undergoing ART. In some embodiments of the invention, the therapy may be provided to any patient undergoing IVF. In some embodiments of the invention the therapy may be provided to patients wherein lower levels of gonadotropins within a particular medical regime are desired or required. In some embodiments of the invention the therapy may be provided to patients wherein lower levels of gonadotropins within an IVF protocol may be necessary, desired or an alternative.

In some embodiments of the invention, the subject may be diagnosed with low ovarian reserves.

Mammals, for example women who are diagnosed with low ovarian reserve levels and/or women who want to extend their childbearing years in advance of a known unnatural decline, and/or a woman undergoing controlled ovarian hyperstimulation may be provided, in some embodiments of this invention, with the pharmaceutical formulation described herein and above. In certain non-limiting embodiments, said mammalian subject may be provided with the IL-1 inhibitor/s therapy of the invention from early stages of puberty. In other non-limiting embodiments, the mammal may be provided with the therapy of the invention at any stage of puberty. A woman, or in some embodiments, a transgender individual who is capable of childbearing, may respond poorly to controlled ovarian hyperstimulation, for example, because they suffer from a pathological condition that results in low ovarian reserve even at a relatively young age. A poor response to controlled ovarian hyperstimulation condition may warrant, in some embodiments of the invention, the providing of the pharmaceutical formulation.

A therapy may be provided for the subject diagnosed with a likelihood of a premature situation of low ovarian reserves, the therapy as described above wherein the dosing is provided subcutaneously on a regular interval.

The providing of the pharmaceutical formulation may be provided at an interval that is dependent both on external factors, such as lifestyle, as well as physiological factors of the woman or the patient, and/or factors related to the nature of the formulation. Some drugs may necessitate a multiple dose daily, a daily injection or an otherwise daily administration of the drug, either actively or passively via a patch or other method, or a weekly, bimonthly, every two months, every quarter of a year, every third of a year, twice a year, once a year or other intervals, either regularly, or in some embodiments of the invention, not regularly.

The providing of the IL-1 inhibitor of the invention or any pharmaceutical formulation or composition thereof may be during the span of child bearing years of an individual, or may be for less than the span of childbearing years of an individual. The providing of the pharmaceutical formulation, in some embodiments of the invention may be near the end of the childbearing age of an individual, at the end of the child bearing age of an individual or after the childbearing age of the individual.

In some embodiments of the invention, the therapy may continue until the childbearing years are over. In some embodiments of the invention, the therapy may continue for as long as the subject wants, or, if the subject is an animal, as long as the owner of the animal wants.

As noted above, the present invention involves the use of different active ingredients, for example, the IL-1 inhibitor used by the invention, and at least one gonadotropin that may be administered through different routes, dosages and combinations. More specifically, the treatment of infertility and related conditions with a combination of active ingredients may involve separate administration of each active ingredient. Therefore, a kit providing a convenient modular format of the IL-1 inhibitor used by the invention and gonadotropins required for treatment would allow the required flexibility in the above parameters.

Thus, in another aspect, the invention provides a kit that may include at least two separate pharmaceutical compositions that are required for modulating an ovarian stimulation.

Thus, in another aspect, the invention provides a kit. In certain embodiments the kit of the invention may comprise: (a) at least one IL-1 inhibitor or any composition or formulation thereof, optionally, in a first dosage form; and (b) at least one gonadotropin or any compound inducing COH, optionally, in a second dosage form.

In certain embodiments, the kit of the invention is particularly applicable for use in a method of any one of, retaining ovarian follicle reserve in a mammalian subject, treating infertility and improving responsiveness of a treated subject to COH medications.

It should be appreciated that each of the multiple components of the kit may be administered simultaneously. Alternatively, each of said multiple dosage forms may be administered sequentially in either order. More specifically, the kits described herein can include a composition as described, or in separate multiple dosage unit forms, as an already prepared liquid injectable, topical or oral dosage form ready for administration or, alternatively, can include the composition as described as a solid pharmaceutical composition that can be reconstituted with a solvent to provide a liquid injectable, or oral dosage form. When the kit includes a solid pharmaceutical composition that can be reconstituted with a solvent to provide a liquid dosage form (e.g., for injection or oral administration), the kit may optionally include a reconstituting solvent. In this case, the constituting or reconstituting solvent is combined with the active ingredient to provide liquid oral dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

More specifically, the kit of the invention may comprise (a) at least one IL-1 inhibitor or any composition or formulation thereof, optionally, in a first dosage form.

The kit of the invention may further comprise (b) at least one gonadotropin/s, and a pharmaceutically acceptable carrier or diluent, optionally, in a second unit dosage form. In such case, the kit may be applicable in enhancing the sensitivity of the treated subject, and of the subject's ovarian cells to said gonadotropin treatment.

According to some embodiments, the kit of the invention may further comprise container means for containing said first and second dosage forms. The term “container” as used herein refers to any receptacle capable of holding at least one component of a pharmaceutical composition of the invention. Such a container may be any jar, vial or box known to a person skilled in the art and may be made of any material suitable for the components contained therein and additionally suitable for short or long term storage under any kind of temperature. More specifically, the kit includes container means for containing separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

It should be appreciated that each of the multiple components of the kit may be administered simultaneously.

Alternatively, each of said multiple dosage forms may be administered sequentially in either order.

As indicated above, some embodiments of the invention, by using the IL-1 inhibitors, optionally with gonadotropins, offer a combined therapy. The phrase “combination therapy” or “adjunct therapy” or in defining use of a compound described herein, specifically, the IL-1 inhibitor or blocker of the invention, and one or more other active pharmaceutical agents, specifically, gonadotropin/s, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

In some embodiments, the diagnostic method of the invention may involve the evaluation of further markers, for example, AMH and FSH.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates, mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Material and Methods

### Mice

The generation of IL-1α and IL-1β knockout (KO) mice on the C57Bl/6 genetic background has been described previously and these mice were generously given to us by Ron N. Apte from Ben-Gurion University of the Negev, Beer-Sheva, Israel. WT C57Bl/6 mice were purchased from Harlan Laboratories, Jerusalem, Israel. Mice were maintained on a 12-h light/12-h dark cycle in the animal facility of the Sackler faculty of Medicine in Tel-Aviv University. Animal care was in accordance with institutional guidelines and was approved by the local authorities (Animal care committee). Both IL-1α-KO and IL-1β-KO exhibits normal development and health without any observed differences compared to their WT counterparts. These mice do not exhibit evidence of spontaneous carcinogenesis and their lifespan appears normal.

### Breeding

WT and IL-1α-KO female mice were individually caged with WT male of proven fertility for a mating period of one month. Female mice were weighed at the beginning of the mating period and 14 days after mating followed by daily weighing. Female mice that gained weight (at least 3 grams) and subsequently exhibited a weight loss were regarded as experiencing a miscarriage and were sacrificed. Otherwise, they were sacrificed close to delivery. Fetuses were classified as viable or nonviable (when resorbed or dead). Pregnancy was defined as having at least one fetus at laparotomy. The number of fetuses per female was recorded.

### Super-Ovulation

WT and IL-1α-KO female mice at various ages were primed with gonadotropins as previously described [Bar-Joseph H et al. (2010)]. Ovulated cumulus-oocyte-complexes were isolated from the oviductal ampullae into M2 medium (Sigma) 16-18 hours after hCG administration. Oocytes were denuded by a brief exposure to 400 IU/ml hyaluronidase (Sigma) and counted.

### Serum AMH Measurement

Blood samples were drawn via the retro-orbital sinus and sera were extracted and kept in −80° until use. Measurements were performed using Enzyme-linked immunoassay (ELISA) according to the manufacture instructions (Beckman Coulter, Inc. 250 S. Kraemer Blvd. Brea, Calif. 92821 U.S.A.). Calculated Inter-assay CV (n=5)=8% and calculated Intra-assay CV (n=37)=3.2%.

### Western Blot Analysis

Ovaries were subjected to western blot analysis as previously described [Eliyahu (2007)].

### Antibodies

Primary antibodies included rabbit anti-IL-1α (sc-7929; Santa Cruz Biotechnology, Santa Cruz, Calif.), goat anti-IL-1α (AF-400-NA, R&D systems, McKinley, Minneapolis), rabbit anti-AMH (sc-28912), mouse anti-Bax (sc-7480), anti-Bcl-2 (SC-783), anti-PARP (SC7150), goat anti-actin (sc-1615), rabbit anti-ki-67 (E1871). Secondary antibodies included Cy3 anti goat and horseradish peroxidase-conjugated monoclonal and polyclonal antibodies (Jackson Immunoresearch, West Grove, Pa.).

### RNA Isolation, RT-PCR and Quantitative Real-Time PCR

Total RNA was isolated from mouse ovaries using a Trizol reagent (Invitrogen), according to manufacturer's instructions, and quantified with the Nano-Drop spectrophotometer (ND-1000; Thermo Scientific). First-strand cDNA was created by RT (Applied biosystems, Foster City, Calif.) from a total of 1 μg RNA using 1 μl of reverse transcriptase. DNA was amplified (35 cycles) with 0.4 μM gene-specific primers using either ready-mix mixture (Sigma) or TaKaRaEx Taq™ (Takara Biotechnology). The PCR products were separated by a 2% agarose gel electrophoresis and visualized by ethidium bromide staining. Changes in the level of mRNA expression were detected using gene specific primers and Fast SYBR Green Master Mix reagent (Applied biosystems) or FastStart Universal probe Master (Roche) along with the appropriate UPL (Universal Prob Library) and 20-50 ngc DNA on a StepOnePlus Real-Time PCR System (Life technologies). The thermal cycling conditions for Fast SYBR Green reaction were 20 sec at 95° C., followed by 40 cycles of 3 sec at 95° C. and 30 sec at 60° C. The thermal cycling conditions for FastStart Universal probe Master were 10 min at 95° C. followed by 40 cycles of 10 sec at 95° C. and 30 sec at 60° C. PCR primers for Probe Library assays were designed with the Probe Library Assay Design Center.

The following primers were used: UPL #29 for amplification of IL-1α and GAPDH, UPL #48 for IL-10 and FSH-R, UPL #78 for IL-1β, UPL #69 for Bax and HPRTI, UPL #2 for BCL-2 and UPL #49 for TNFα. The specific primer sequences used are disclosed in Table 1.

TABLE 1
Specific PCR and qPCR Primers

| | | SEQ |
|---|---|---|
| Primers for PCR | Sequence (5′-3′) | ID NO: |
| IL-1α | | |
| Forward | TTGGTTGAGGGAATCATTCAT | 1 |
| Reverse | TCCATAACCCATGATCTGGAA | 2 |
| IL-1β | | |
| Forward | GTGGCAGCTACCTGTGTCT | 3 |
| Reverse | GAGCCTGTAGTGCAGTTGTCT | 4 |
| IL-1RI | | |
| Forward | CTCTGCTGTCGCTGGAGATT | 5 |
| Reverse | GAAGGTGGCCTGTGTGC | 6 |
| GAPDH | | |
| Forward | GTGAAGGTCGGTGTGAACGG | 7 |
| Reverse | GTGATGGCATGGACTGTGGTC | 8 |
| Primers for qPCR | | |
| FSHR | | |
| Forward | AGTGTTTAATGCCTGTGTTGGA | 9 |
| Reverse | ACCCTGAGGCCTTCCAGA | 10 |
| Bax | | |
| Forward | GCTGAGCAGGGTCTTCAGAG | 11 |
| Reverse | GAACCATCATGGGCTGGA | 12 |
| Bcl-2 | | |
| Forward | GCTGAGCAGGGTCTTCAGAG | 13 |
| Reverse | GTACCTGAACCGGCATCTG | 14 |
| IL-6 | | |
| Forward | AGCCAGAGTCCTTCAGAG | 15 |
| Reverse | CCACTCCTTCTGTGACTC | 16 |
| IL-10 | | |
| Forward | GTTGTCCAGCTGGTCCTTTG | 17 |
| Reverse | CAGCCGGGAAGACAATAACT | 18 |
| TNF-α | | |
| Forward | GAGGCCATTTGGGAACTTCT | 19 |
| Reverse | TGCCTATGTCTCAGCCTCTTC | 20 |
| IL-111 | | |
| Forward | TCTTCTTTGGGTATTGCTTGG | 21 |
| Reverse | TGTAATGAAAGACGGCACACC | 22 |
| HPRT1 | | |
| Forward | CTGGTTCATCATCGCTAATCAC | 23 |
| Reverse | GGAGCGGTAGCACCTCCT | 24 |
| GAPDH | | |
| Forward | GTGAAGGTCGGTGTGAACGG | 7 |
| Reverse | GTGATGGCATGGACTGTGGTC | 8 |

### Morphometric Analysis

Ovaries were collected from WT and IL-1α-KO female mice at the indicated ages and fixed in Bouin's solution. Paraffin embedded ovaries were serially sectioned (8 μm) and stained with H&E. In every fifth ovarian section the number of primordial, primary, secondary and antral follicles was counted. Follicles were classified as previously described. In aged mice (12 month) analysis was done in every second ovarian section and only follicles containing an oocyte with a visible nucleus were counted to avoid double counting. The results are reported as the number of counted follicles per ovary.

### Immunofluorescence

Ovaries were collected from WT and IL-1α-KO mice at the indicated age, fixed in 4% paraformaldehyde (PFA) and embedded in paraffin. Ovarian sections (6 μm) were stained as previously described. Primary GCs were extracted and plated on 13-mm round glass coverslips in DMED-F12 medium supplemented with 10% FBS. After 24 hours, cells were fixed for 30 minutes in 4% paraformaldehyde and permeabilized for 1 minute with 0.02% Triton X-100 in PBS. Cells were subsequently incubated at room temperature with goat anti-IL-1α for 1.5 hours, washed 3 times in PBS and incubated with the appropriate secondary antibodies together with Hoechst 33342 for DNA labeling for additional 45 min. GV oocytes were isolated into M2 medium (Sigma) supplemented with 1 μM Milrinon (Sigma) to avoid oocyte maturation. Cumulus cells were removed mechanically and oocytes were stained. All experimental samples were visualized and photographed using a Leica laser confocal microscope (SP5; Leica, Wetzlar, Germany) that was calibrated to a secondary-only control.

### Anakinra Treatment

Mice were injected daily with 0.5 mg Anakinra (IL-1 receptor antagonist) or saline (control) for 30 days (n=18 in each group). Eight mice from each group were primed with 71U PMSG, their GCs isolated 48 hours later, cultured in a 24 well plate, collected 4 hours later and subjected to RNA extraction for the detection of FSHR. Raw data were normalized to GAPDH expression and fold change relative to control was calculated. The remaining 10 mice from each group were primed with PMSG and hCG, as previously described [Bar-Joseph H (2010)]; the number of ovulated oocytes was recorded 16 hours later. Oocytes recovered from 5 out of these 10 mice were fertilized in vitro by WT sperm; the percentage of 2-cell embryos was recorded 24 hours later for each mouse genotype.

### In Vitro Fertilization

WT and IL-1α-KO female mice were primed with gonadotropins as described above. Spermatozoa were isolated from the cauda epididymis of WT mice into human tubal fluid (HTF, self-made) medium and allowed to capacitate for 2 hours at 37° C., 5% CO2 in air. At the end of the capacitation period, ovulated cumulus-oocyte complexes were isolated into drops of 150 μl of capacitated spermatozoa (1.5×10⁶/ml) under oil and incubated for 24 hours to allow fertilization. The presence of 1-cell zygotes and 2-cell embryos was recorded at the end of the incubation period.

### Statistical Analyses

The values reported are the mean±SE. Student's t-test was used as applicable. Mann-Whitney test was used to assess differences between means when the variable tested was not normally distributed. Fisher's exact test or chi² were used as appropriate. The strength of the association between IL-1α or IL-1β and age used as continuous variables was tested by Pearson correlation test. p<0.05 is accepted as statistically significant. All experiments were repeated at least three times with similar results.

### Example I

### IL-1α Deficiency Increases Pregnancy Rate and Mean Litter Size in Advanced Chronological Age

The present invention stems from the initial observation that pregnancy rates are markedly greater in the aged (10-12 month old) IL-1α-knock out (KO) compared to wild type (WT) mice (36/41 (88%) and 23/38 (60%) respectively, p=0.009). A sub-analysis according to age revealed that pregnancy rates were greater both in 10 and 12 month old IL-1α-KO compared to WT mice (Table 1). Importantly, although mean litter size was similar at 10 months of age, there was a significantly larger litter size in 12 month old IL-1α-KO compared to WT mice.

More specifically, the results in Table 2 illustrate one of the main aspects of the present invention being that IL-1α deficiency increases pregnancy rates and mean litter size in advanced chronological age. To demonstrate this aspect, female mice of both genotypes at the indicated age were individually caged with WT male of proven fertility for a mating period of one month. Pregnancy rates and mean litter size were recorded at day 15-18 post conception. Table 2 shows data represented as mean±SE. The number of mice per group is indicated. Statistical significance was determined using Fisher's exact test and Mann-Whitney test.

TABLE 2
Pregnancy rate and mean litter size
in aged WT and IL-1α-KO mice

| 12 months | | 10 months | | |
|---|---|---|---|---|
| IL-1α-KO | WT | IL-1α-KO | WT | |
| 81% | 53% | 95% | 67% | Pregnancy rate |
| n = 21 | n = 17 | n = 20 | n = 21 | |
| 0.087 | | 0.045 | | P-value |
| 3.6 ± 0.6 | 1.0 ± 0.5 | 4.8 ± 0.8 | 4.9 ± 0.7 | Mean litter size |

| | | |
|---|---|---|
| 0.009 | 0.808 | P-value |

Further, the effect of IL-1, being the second major IL-1 prototypic agonist, was evaluated on reproduction lifespan by comparing pregnancy rate of 12 month old WT (n=17) and IL-1β-KO (n=18) mice. While pregnancy rate in IL-1β-KO mice was not statistically different compared to WT (13/18 (72%) and 9/17 (53%) respectively, p=0.2), the mean viable litter size was significantly larger in IL-1β-KO compared to WT (3.4±0.7 and 1.0±0.5 respectively, p=0.017), similar to that observed in IL-1α-KO mice.

### Example I

### Reproductive Advantage of IL-1α-KO Mice is First Manifested in the Middle of the Reproductive Life

To elucidate the cause underling the reproductive advantage of IL-1α-KO mice in advanced age, WT and IL-1α-KO mice were compared with respect to the total follicle number in ovarian serial sections at the age of 1.5, 2.5 and 12 month old. It was found that both genotypes were not significantly different by the total number of follicles at these time points, but by the distribution of types of follicles as PMF (primordial follicles), P (primary follicles), S (secondary follicles), A (antral follicles) (FIG. 1C to 1E).

Thus, FIGS. 1A to 1E illustrate various embodiments of the present invention. FIG. 1A refers to the mean total number of follicles in ovaries as meaningful reproductive markers, exemplified here by the mean number of follicles in ovaries of 1.5, 2.5 and 12 months old WT and IL-1α-KO (αKO) mice. FIG. 1B refers to representative ovarian section micrographs, exemplified here in 2.5 months old WT and IL-1α-KO mice. FIG. 1C refers to the number of ovarian follicles of the indicated types at a pubertal young age, exemplified in 1.5 months WT and IL-1α-KO mice. FIG. 1D refers to the number of ovarian follicles of the indicated types at a middle reproductive age, exemplified in 2.5 months in WT and IL-1α-KO mice. FIG. 1E shows the number of ovarian follicles of the indicated types at an aged ovary (an old reproductive age), exemplified in 12 months in WT and IL-1α-KO mice. For demonstrating these embodiments, the number of each different type of follicle was assessed in every fifth serial section of 1.5 months or 2.5 months respectively of WT and IL-1α-KO mice (FIGS. 1C and 1D) and in every second serial section of ovaries from 12 month old WT and IL-1α-KO mice (FIG. 1E). In FIGS. 1C to 1E, data represent mean±SE in groups of mice (n) as in FIG. 1A. Statistical significance was determined using student's t-test comparing IL-1β-KO to WT considering *P<0.05 as significant.

Specifically, a sub-analysis of follicle number while accounting for a developmental stage revealed no difference in the number of primordial, primary, secondary and antral follicles in 1.5 and 12 months old IL-1α-KO and WT ovaries (FIGS. 1C and 1E). However, the number of secondary and antral follicles was significantly higher in 2.5 month old IL-1α-KO compared to WT ovaries (FIG. 1D) along with a trend towards a higher number of primary follicles (p=0.15). Representative images of 2.5 month old WT and IL-1α-KO ovarian sections are shown in FIG. 1B.

Thus, the association of IL-1α deficiency to higher pregnancy rate and higher viable litter size in advanced chronological age shown herein above in Example I is further supported by the present association of IL-1α deficiency to a higher number of growing follicles at that age. Importantly, the reproductive advantage of IL-1α-KO mice is first manifested at a middle reproductive age and may last up to an advanced age, exemplified in mice at 2.5 and 12 months of age. More specifically, the similar ovarian follicular pool 1.5 months after birth, which has been previously shown also by others [Morita et al., (2000)], suggests that IL-1α-KO and WT mice are endowed with a comparable number of germ cells. However, the findings of the invention is that 2.5 months after birth, ovaries from IL-1α-KO mice contain a significantly higher number of growing follicles compared to WT mice, strongly support the notion that IL-1α deficiency is associated with dramatically more pronounced response to super-ovulation at 2.5 months and up to one year, as well as a higher number of growing follicles.

### Example III

### IL-1α-Deficiency Results in a Better Ovarian Response to Gonadotropins Throughout the Reproductive Lifespan

The ovarian response to super-ovulation (priming with gonadotropins), as an indicator of the growing follicular pool, was further assessed by the inventors in WT and IL-1α-KO mice. FIG. 2A shows that while at 1.5 month the number of oocytes retrieved from the oviducts was similar in both the WT and IL-1α deficient genotypes, the oocyte number at 2.5 months was markedly higher in the IL-1α-deficient (IL-1α-KO) compared to WT mice and this advantage continued throughout the reproductive lifespan until one year.

More specifically, FIGS. 2A to 2B illustrate various embodiments of the present invention showing that IL-1α deficiency results in an augmented response to gonadotropins starting from a middle reproductive age (e.g. mice at the age of 2.5 months) manifested in higher number of oocytes collected from oviducts after gonadotropin priming and continuing throughout the reproductive lifespan. For this purpose, WT and IL-1α-KO (αKO) mice at indicated ages (7-11 mice in each group) were primed with 10 IU of human chorionic gonadotropins (HCG, an LH analog) 48 hours after pregnant mare serum gonadotropin (PMSG, an FSH analog) administration (7 IU). The number of oocytes collected from the oviducts was recorded 16 hours after HCG administration. Data are presented as means±SE and compared using student's T-test with *P<0.05 considered significant.

Further, FIG. 2B includes data from an analogous experiment in IL-1β-KO and in IL-1 Receptor type 1 knockout (IL-1R1-KO) mice at the age of 2.5 months, showing that IL-1β-KO mice ovulated similar to IL-1α-KO mice i.e. with a significantly higher number of oocytes than WT mice (52.8±5.5 and 27.4±4.6 respectively, p<0.005). The response of IL-1R1-KO mice was similar to WT.

It should be noted that the IL-1R1 expression in mice was localized within oocytes and granulosa cells throughout follicular development. IL-1α and IL-1β mRNAs were detected in granulosa cells in response to lipopolysaccharide (LPS). However, IL-1α or IL-1β mRNAs and protein were not previously shown to be expressed within oocytes and/or GCs during follicular development, it is only after follicular rupture that these proteins were observed in these cells. The present findings originally demonstrate the expression of both IL-1α and IL-1β mRNA in oocytes and granulosa cells of developing follicles and further demonstrate the localization of IL-1α protein within oocytes and granulosa cells throughout follicular development.

Further, FIG. 3A shows mean FSHR mRNA expression levels in GC in WT and IL-1α-KO prepubertal (1 month) and pubertal (3 months) mice, constituting yet another embodiment. FSHR is expressed by GCs of the growing follicles and its stimulation following FSH secretion promotes follicular growth [George J W, (2011)]. Specifically, this figure shows that IL-1α deficiency results in higher FSHR expression in GCs in the pre-pubertal as well as pubertal age. For this embodiment, GCs were isolated from ovaries of WT and IL-1α-KO mice and seeded on a 24 well plate. Following adhesion (4 hours), cells were collected and subjected to RNA extraction for the detection of FSHR in pre-pubertal or pubertal mice using qPCR. Raw data were normalized to GAPDH expression and fold change was calculated relative to WT. Data represent mean±SE. Statistical significance was determined using student's T test, with *P<0.05 considered significant.

Still further, FIG. 3B shows FSHR transcript levels in IL-1α-KO ovaries compared to WT following ovarian stimulation with gonadotropins, which is another specific embodiment. Specifically, this figure shows that ovarian FSHR mRNA levels 24 hours after PMSG stimulation were significantly higher in IL-1α-KO compared to WT mice, while at 48 hours—were higher but not statistically significant. For this application, RNA was extracted from ovaries of 2.5 months old WT and IL-1α-KO mice were excised 24 and 48 hours after PMSG administration and subjected to qPCR analysis for the detection of FSHR as described above. Data is presented as mean±SE and analyzed by student's t-test (n=4-5 in each group) with *P<0.05 considered significant.

### Example IV

### Anti Mullerian Hormone (AMH) Levels are Higher in IL-1α-KO Compared to WT Mice

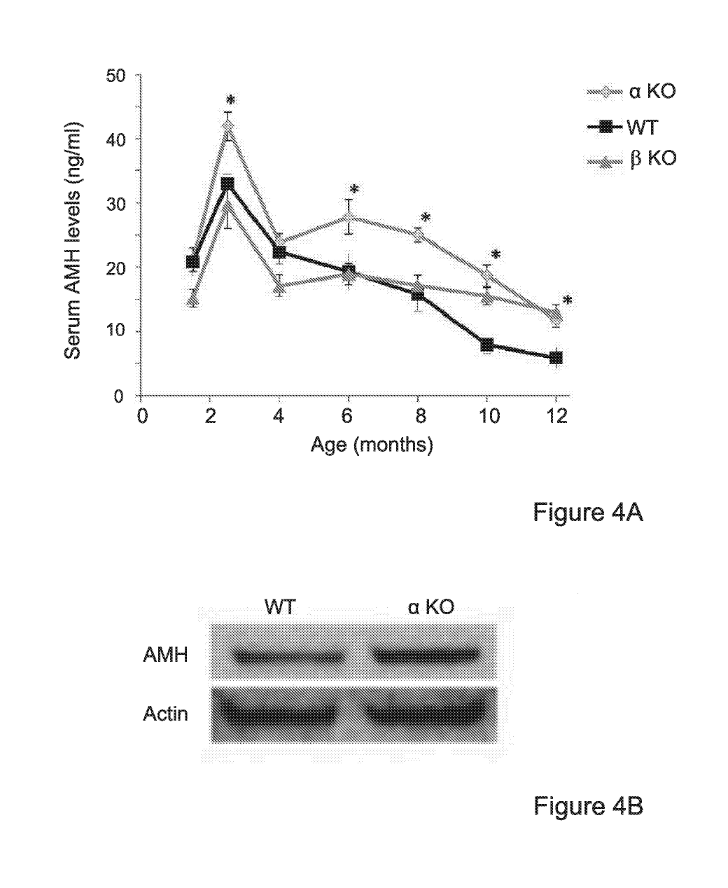

Further, ELISA was used to measure serum AMH levels as a putative marker of the ovarian reserve. Serum AMH levels were similar in 1.5 month old WT and IL-1α-KO mice and rose in both strains reaching a peak at 2.5 months after birth. FIG. 4A shows that IL-1α-KO mice have higher serum AMH compared to WT which constitutes a specific embodiment of the invention. For this purpose, mean serum AMH levels in WT, IL-1α-KO (αKO) and IL-1β-KO (βKO) mice (n=9-11 in each group) were measured. At the indicated age, blood was drawn from the retro-orbital sinus and measurement of serum AMH was performed using Beckman Coulter Enzyme-linked immunoassay (ELISA).

Further, FIG. 4B shows that IL-1α-KO mice have higher ovarian AMH protein level compared to WT, thus constituting a further embodiment. For this purpose, protein lysates of ovaries from 2.5 months old WT and IL-1α-KO mice (n=5 for each group) were pooled and subjected to western blot analysis for the detection of AMH. Data represent mean±SE. Statistical significance was determined using student's t-test, with *P<0.05 compared to WT considered significant.

It should be noted that the serum AMH levels at this time point were markedly higher in IL-1α-KO compared to WT mice and this advantage was maintained throughout the reproductive lifespan (FIG. 4A). Interestingly, in advanced chronological age, the serum AMH levels were higher also in IL-1β-KO mice compared to WT mice (FIG. 4A). In accordance with serum levels, ovarian AMH protein levels were higher in 2.5 month old IL-1α-KO compared to WT mice as determined by Western blotting (FIG. 4B).

Still further, in both strains a post pubertal ascent in serum AMH was observed. However, the AMH peak at 2.5 months was markedly higher in IL-1α-KO compared to WT and this advantage was maintained throughout the reproductive lifespan. Furthermore, the mRNA levels of FSHR, which is similarly to AMH expressed by growing follicles, were also higher in IL-1α-KO compared to WT ovaries. Taken together, these findings indicate that from 2.5 months onwards, IL-1α-KO mice contain a larger population of viable growing follicles both at the basal state and after PMSG priming. Lack of significantly higher number of follicles in IL-1α-KO mice at 12 months may be explained by the small sample size. In aged mice, serum AMH levels reflect the size of the resting follicular pool. Therefore, higher AMH levels in advanced age suggests a larger pool of resting follicles in IL-1α-KO compared to WT mice. As AMH is known to inhibit the recruitment of the non-growing follicles, higher serum AMH levels from 2.5 months onwards could be related to a larger non growing follicular pool suggested in IL-1α-KO mice.

### Example V

### The Expression Pattern of IL-1α, IL-1β and IL-1R1 within the Ovary

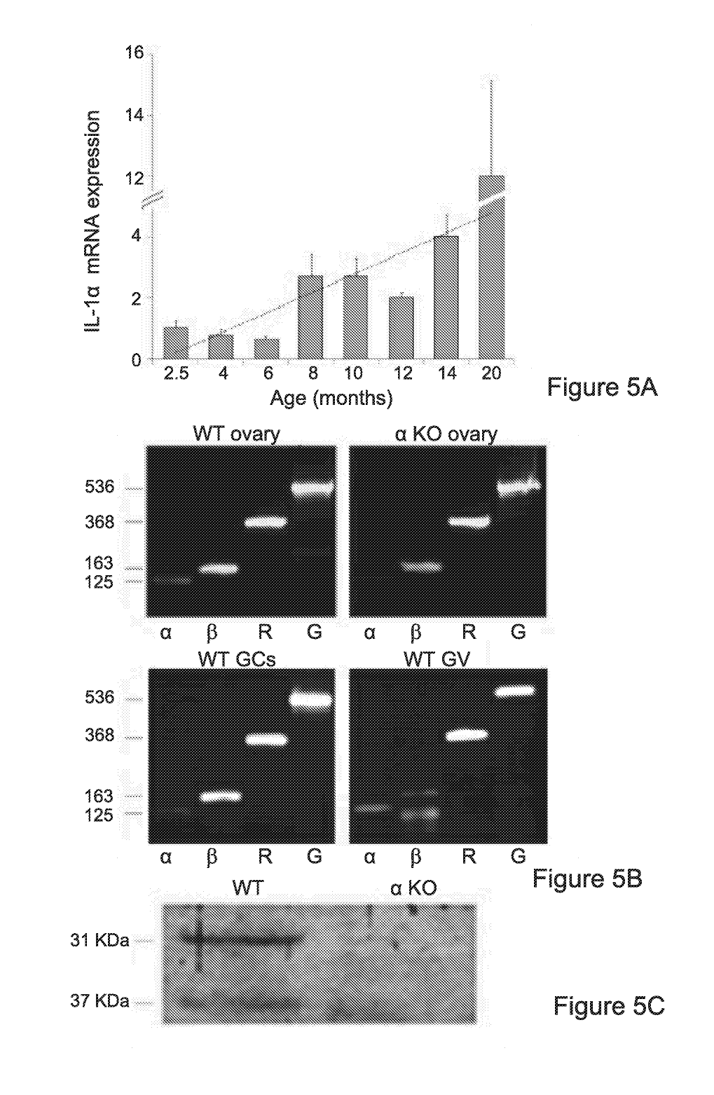

The ovarian mRNA levels of IL-1α as measured by quantitative polymerase chain reaction (qPCR) were positively correlated with age, which is another specific embodiment. This was also true for the ovarian IL-1β levels (data not shown). FIG. 5A shows the expression of IL-1α mRNA in the ovary of WT mice during aging (starting at 2.5 months and up to 20 months). The expression of IL-1α mRNA was analyzed using qPCR and normalized to the expression level measured at 2.5 months. Pearson Correlation was estimated at 0.66. The dotted line in the figure represents the trend of the IL-1 expression.

FIG. 5B shows PCR analysis of RNA extracted from ovaries of WT and IL-1α-KO mice and from GCs and GV oocytes of WT mice, constituting a specific embodiment of the invention. For this purpose, RNA extracted from ovaries of WT and IL-1α-KO (αKO) mice and from GCs and GV oocytes of WT mice, was subjected to PCR analysis for detection of IL-1α (α, 125 bp), IL-1β (b, 163 bp) and IL-1R1 (R, 368 bp) mRNA transcripts. GAPDH (G, 536 bp) is indicated as internal control. FIG. 5C shows a western blot of protein lysates of ovaries from WT and IL-1α-KO mice, yet a further embodiment. For this purpose, protein lysates of ovaries from WT and IL-1α-KO mice (n=5 for each group) were pooled and subjected to western blot analysis for detection of IL-1α.

In summary, the increase in IL-1α with age was more prominent (Pearson Correlation of 0.66 and 0.55 in IL-1α and IL-1β respectively, p<0.001, FIG. 5A). IL-1α, IL-1β and IL-1RI mRNA transcripts were present in whole ovary, in GCs and in GV oocytes from WT mice (FIG. 5B), while IL-1α mRNA was absent in ovaries from IL-1α-KO mice (αKO, negative control). The presence of IL-1α protein within the ovary was validated using western blot analysis (FIG. 5C) and its localization in GCs and oocyte was demonstrated using immunofluorescence.

Further, FIG. 6 shows stained paraffin sections of IL-1α protein in GCs and oocytes of WT ovaries, which is a specific embodiment. For this purpose, paraffin embedded ovarian sections from 6 days (FIG. 6A) and 2.5 month old (FIGS. 6B-6E) WT mice and isolated primary GCs (FIG. 6F) and GV oocytes (FIG. 6G) from 2.5 month old WT mice were stained with anti-IL-1α antibody and Hoechst for DNA labeling. The figure shows the presence of the IL-1α protein in the cytoplasm of GCs (arrows) and oocytes (asterisk) throughout follicular development. Negative control using secondary antibody alone is demonstrated in micrographs Section H (paraffin embedded ovarian section) and Section I (isolated oocyte). Scale bar is indicated at each micrograph.

Although GCs from PMFs are difficult to discern, the IL-1α protein is clearly seen in the cytoplasm of GCs from primary follicles and it is expressed throughout the follicular development up to the late antral stage. The presence of IL-1α protein within the oocyte is prominent during all the stages of follicular development. It is mainly localized in the cytoplasm and to a lesser extent in the GV of the oocyte.

### Example VI

### Increased Proliferation of IL-1α-Deficient GCs

Next, the inventors examined the effect of IL-1α deficiency on the proliferative ability of ovarian GCs. Therefore, the number of proliferating GCs in IL-1α-KO and WT ovarian sections was qualitatively compared using IHC staining with Ki-67 antibody. This monoclonal antibody detects a nuclear structure present exclusively in proliferating cells and is therefore extensively used as a proliferation marker [Gerdes J, (1983); Scholzen T, (2000)]. FIG. 7 shows that the number of proliferating GCs was significantly higher in follicles of IL-1α-KO than in WT mice, representing another specific embodiment. For this purpose, representative sections of paraffin embedded ovaries of 2.5 month old WT (FIG. 7A) and IL-1α-KO (FIG. 7B) mice were stained with the monoclonal Ki-67 antibody (dark signal). A larger magnification of the indicated areas in micrographs of FIGS. 7A and 7B are presented in micrographs of FIGS. 7C and 7D, respectively. Ki-67 staining is evident in follicular GCs (arrows). Positive control of Ki-67 staining is shown in micrographs (FIG. 7E, tonsil). The scale bar (100 micrometer) indicated in micrograph of FIG. 7B relates to micrographs of FIGS. 7A and 7B.

### Example VII

### Apoptotic Proteins and Transcripts of Inflammation-Related Genes are Lower in IL-1α Deficient Mice

Further, the inventors assessed whether IL-1α deficiency renders ovarian cells less vulnerable to apoptosis during follicular development. FIG. 8A shows a western blot of a protein lysates of ovaries from 2.5 months old WT and IL-1α-KO mice. Specifically, the figure shows that expression of pro-apoptotic proteins (FIG. 8A) and inflammation related mRNA levels (FIG. 8B) are lower in ovaries from IL-1α-KO compared to WT mice, thus constituting another specific embodiment. For this purpose, protein lysates of ovaries from 2.5 months old WT and IL-1α-KO mice (n=5 for each group) were pooled and subjected to western blot analysis for the detection of the pro-survival protein Bcl-2, the pro-apoptotic Bax and the cleaved form of PARP (cPARP). As shown in FIG. 8A, the expression of the pro-apoptotic protein Bax and the cleaved form of PARP were substantially lower, while the anti-apoptotic protein Bcl-2 was markedly higher in IL-1α-KO compared to WT ovaries.

The question whether IL-1α deficiency affects apoptosis-related pathways in the ovary is particularly important in view of an apparent lack of information on the role of IL-1α in apoptosis and its involvement in apoptosis within the ovary in particular. In the mammalian ovary, more than 99% of follicles are destined to atretic degeneration (an apoptotic process). Follicular atresia is mediated via the engagement of death ligands (e.g. TNFα and Fas) to plasma membrane death receptors, or through the mitochondrial pathway within the cell in which the Bcl-2 family members play an important role. Nevertheless, the molecular mechanisms responsible for the delicate balance to determine life and death of cells in the ovary are not well understood.

FIG. 8B shows a qPCR analysis for the detection of TNFα, IL-6, IL-1β, IL-10 from RNA extracted from ovaries of 2.5 month old WT and IL-1α-KO mice, yet another specific embodiment. For this purpose, RNA was extracted from ovaries of 2.5 month old WT and IL-1α-KO mice and subjected to qPCR analysis for the detection of TNFα, IL-1β, IL-6 and IL-10. Raw data are normalized to GAPDH expression and fold change was calculated relative to WT. n=3-9 in each group. Data represent mean±SE. Statistical significance was determined using student's T test, with *p<0.05 considered significant. The results presented in FIG. 8B show that TNFα as well as IL-1β, IL-6 and IL-10 were markedly lower in ovaries from IL-1α-KO compared to WT mice.

In summary, IL-1α deficiency resulted in a higher expression of the anti-apoptotic Bcl-2 protein and lower levels of the pro-apoptotic proteins Bax and PARP within the ovary. The Bcl-2 family members Bcl-2 and Bax were found mainly in developing and atretic follicles, respectively. IL-1α deficiency affected neither Bax nor Bcl-2 mRNA levels, indicating their regulation at the protein rather than at mRNA level. Furthermore, the cell death inducing cytokine TNFα, which was previously shown to induce apoptosis within the ovary, was 3.5 fold lower in IL-1α-KO compared to WT ovaries. Taken together, these findings suggest that IL-1α promotes apoptotic signaling within the ovary both by the extracellular TNFα pathway and the intracellular mitochondrial pathway.

Further, IL-1α deficiency resulted in reduced ovarian expression of pro-inflammatory cytokines other than TNFα, such as IL-1β and IL-6. The lower mRNA levels of the anti-inflammatory cytokine IL-10 can be attributed to a decreased necessity to counteract the inflammatory milieu which is attenuated in IL-1α-KO. These findings are particularly important as inflammatory conditions were related to poor IVF outcomes. Endometriosis, a chronic inflammatory condition was associated with a poor ovarian response to gonadotropins and lower fertilization and pregnancy rates compared to unaffected IVF patients. Elevated levels of TNFα in the follicular fluid were correlated with poor oocytes quality and TNFα blockage was shown to improve IVF outcomes in young infertile women. Recent studies showing that patients with endometriosis and Crohn's disease have significantly lower AMH levels compared to healthy controls, supporting the hypothesis that uncontrolled inflammation adversely affects ovarian reserve. However, at present no direct solid connection links specific inflammation-related genes with ovarian longevity. The extended reproductive lifespan observed in mice lacking IL-1 strengthens the possible relevance of attenuated inflammatory milieu in maintenance of ovarian reserve.

Still further, findings that both IL-1α and IL-1β transcripts in the ovary increase with age are particularly interesting, as aging was previously characterized by an overall increase in organ IL-1 expression. The age related increase in systemic inflammation is responsible for several age related pathologies in which IL-1 is thought to play an important role. The age related increase in IL-1α expression was shown in endometrial stromal fibroblasts, human endothelial cells and fibroblasts. Moreover, IL-1α accelerates the increase in other age related transcripts. Taken together, the observation of the present invention that IL-1α expression increases with age in normal ovarian tissue also supports its involvement in ovarian senescence.

Further, particularly lower mRNA levels of IL-1β were found in ovaries from IL-1α-KO mice. The mutual induction of IL-1α and IL-1β has been previously documented. IL-1α deficiency was shown to result in reduced expression of IL-1α in macrophages and in the liver. Importantly, IL-1β-KO mice revealed a more subtle but a similar phenotype of reproductive advantage as seen in IL-1α-KO mice. These findings in IL-1β deficient mice suggest that lower expression levels of IL-1β contributed at least in part to the prolonged ovarian life-span observed in IL-1α-KO mice.

### Example VII

### IL-1 Blockade with Anakinra Results in Higher FSHR Transcripts in Mouse GCs

Finally, FIG. 9 shows the effect of IL-1 blockade as examined on FSHR expression levels and oocyte count in mouse primary GCs, which constitutes one of the important aspects of the present invention. For this purpose, WT mice were subcutaneously injected with a daily dose of 0.5 mg Anakinra (recombinant human IL-1 receptor antagonist) for 30 days. FIG. 9A shows that FSHR mRNA levels were significantly higher in GCs of Anakinra-treated mice than in GCs of control mice. FIG. 9B shows that the ovarian response to gonadotropins, as manifested by the number of ovulated oocytes, was comparable in both groups.

More importantly, FIG. 9C shows the outcomes of in vitro fertilization in the treated and untreated WT mice. Specifically, the rate of oocytes fertilization in vitro, as indicated by the number of 2-cell embryos, showed a higher tendency (p=0.15) in Anakinra-treated than WT mice.

A general outline of this preliminary study is shown in FIG. 10. More specifically, mice were injected daily with 0.5 mg Anakinra or saline (control) for 30 days (n=18 in each group). Eight mice from each group were primed with 7IU PMSG, their GCs isolated 48 hours later, cultured in a 24 well plate, collected 4 hours later and subjected to RNA extraction for the detection of FSHR. Raw data were normalized to GAPDH expression and fold change relative to control was calculated (FIG. 9A). The remaining 10 mice from each group were primed with PMSG and hCG, the number of ovulated oocytes was recorded 16 hours later (FIG. 9B). Oocytes recovered from 5 out of these 10 mice were fertilized in vitro by WT sperm; the percentage of 2-cell embryos was recorded 24 hours later for each mouse genotype (FIG. 9C). Data are presented as mean±SE and analyzed by student's T-test.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1-44. (canceled)

45. A method for retaining ovarian follicle reserve in a mammalian subject by blocking, inhibiting or reducing the activity of Interleukine-1 (IL-1), said method comprising the step of administering to said subject an effective amount of at least one IL-1 inhibitor or any composition or formulation thereof.

46. The method according to claim 45, wherein said method results in at least one of:
(a) reduction in ovarian follicular atresia, thereby retaining ovarian follicle reserve in said subject; and
(b) increased expression of follicular stimulating hormone receptor (FSHR) in cells comprised within ovarian follicles of said subject.

47. The method according to claim 45, wherein said IL-1 inhibitor blocks, inhibits or reduces the activity, expression, stability, post-transcriptional or posttranslational processing of at least one of IL-1α, IL-1β, Interleukin-1 receptor type 1 (IL-1R1) or of any molecule participating in signal transduction pathways mediated by IL-1.

48. The method according to claim 47, wherein said IL-1 inhibitor is a synthetic or natural protein compound, peptidomimetic compound, nucleic acid compound, a small molecule or any combinations thereof, and wherein said protein compound inhibitor is an antibody specifically directed against at least one of IL-1α, IL-1β or IL-1R1.

49. The method according to claim 45, for improving oocyte quality in said subject.

50. The method according to claim 45, for improving response of said mammalian subject to a controlled ovarian hyper stimulation (COH).

51. The method according to claim 50, wherein said subject is diagnosed or classified as a poor ovarian responder (POR).

52. The method according to claim 51, wherein said COH comprises administration of at least one gonadotropin and wherein said IL-1 inhibitor or any formulation or composition thereof is adapted for use before, simultaneously with, after or any combination thereof with said at least one gonadotropin.

53. The method according to claim 52, wherein said subject is undergoing an in vitro fertilization (IVF) treatment.

54. The method according to claim 45 for treating, inhibiting, preventing or ameliorating infertility or infertility-related conditions in a mammalian subject.

55. The method according to claim 54, wherein said subject is treated with COH medications.

56. The method according to claim 55, wherein said subject is diagnosed or classified as a POR.

57. The method according to claim 55, wherein said COH medications comprise at least one gonadotropin, and wherein said IL-1 inhibitor or any formulation or composition thereof is adapted for use before, simultaneously with, after or any combination thereof with said at least one gonadotropin.

58. The method according to claim 54, wherein said subject is undergoing an IVF treatment.

59. The method according to claim 45, for sensitizing and increasing responsiveness of ovarian follicular cells to gonadotropin/s treatment.

60. A method of improving oocyte quality and/or survival, the method comprising ex-vivo contacting an oocyte in a cell culture with an effective amount of at least one IL-1 inhibitor that blocks or inhibits IL-1 activity or any composition or formulation thereof, thereby improving oocyte quality and/or survival.

61. The method according to claim 60, wherein said oocyte is maintained under conditions for IVF.

62. The method according to claim 60, wherein said oocyte is comprised in an ovary or an ovarian tissue.

63. A kit comprising:
(a) at least one IL-1 inhibitor or any composition or formulation thereof, optionally, in a first dosage form; and
(b) at least one gonadotropin or any compound inducing COH, optionally, in a second dosage form.

64. The kit according to claim 63, for use in a method of any one of, retaining ovarian follicle reserve in a mammalian subject, treating infertility and improving responsiveness to COH.


SEQUENCE LISTING (63 sequences)

SEQ ID NO: 1 - DNA, 21 bp, Artificial Sequence
  Forward primer (Human Interleukin-1 alpha )
ttggttgagggaatcattcat

SEQ ID NO: 2 - DNA, 21 bp, Artificial Sequence
  Reverse primer (Human Interleukin-1 alpha )
tccataacccatgatctggaa

SEQ ID NO: 3 - DNA, 19 bp, Artificial Sequence
  Forward primer (Human Interleukin-1 beta )
gtggcagctacctgtgtct

SEQ ID NO: 4 - DNA, 21 bp, Artificial Sequence
  Reverse primer (Human Interleukin-1 beta)
gagcctgtagtgcagttgtct

SEQ ID NO: 5 - DNA, 20 bp, Artificial Sequence
  Forward primer (Human Interleukin-1 receptor type )
ctctgctgtcgctggagatt

SEQ ID NO: 6 - DNA, 17 bp, Artificial Sequence
  Reverse primer (Human Interleukin-1 receptor type 1)
gaaggtggcctgtgtgc

SEQ ID NO: 7 - DNA, 20 bp, Artificial Sequence
  Forward primer ( Human Glyceraldehyde 3-Phosphate Dehydrogenase (GAPDH, G3PDH)
gtgaaggtcggtgtgaacgg

SEQ ID NO: 8 - DNA, 21 bp, Artificial Sequence
  Reverse primer (Human Glyceraldehyde 3-Phosphate Dehydrogenase (GAPDH, G3PDH)
gtgatggcatggactgtggtc

SEQ ID NO: 9 - DNA, 22 bp, Artificial Sequence
  Forward primer  (Human Follicle-Stimulating Hormone Receptor (FSH receptor, FSHR)
agtgtttaatgcctgtgttgga

SEQ ID NO: 10 - DNA, 18 bp, Artificial Sequence
  Reverse primer (Human Follicle-Stimulating Hormone Receptor (FSH receptor, FSHR)
accctgaggccttccaga

SEQ ID NO: 11 - DNA, 20 bp, Artificial Sequence
  Forward primer  (Human BCL2-associated X protein (BAX)
ggtcccgaagtaggagagga

SEQ ID NO: 12 - DNA, 18 bp, Artificial Sequence
  Reverse primer  (Human BCL2-associated X protein (BAX)
gaaccatcatgggctgga

SEQ ID NO: 13 - DNA, 20 bp, Artificial Sequence
  Forward primer  (Human B-cell lymphoma 2 (Bcl-2)
gctgagcagggtcttcagag

SEQ ID NO: 14 - DNA, 19 bp, Artificial Sequence
  Reverse primer  (Human B-cell lymphoma 2 (Bcl-2)
gtacctgaaccggcatctg

SEQ ID NO: 15 - DNA, 18 bp, Artificial Sequence
  Forward primer (Human Interleukin 6 (IL-6)
agccagagtccttcagag

SEQ ID NO: 16 - DNA, 18 bp, Artificial Sequence
  Reverse primer  (Human Interleukin 6 (IL-6)
ccactccttctgtgactc

SEQ ID NO: 17 - DNA, 20 bp, Artificial Sequence
  Forward primer  (Human Interleukin 10 (IL-10)
gttgtccagctggtcctttg

SEQ ID NO: 18 - DNA, 20 bp, Artificial Sequence
  Reverse primer (Human Interleukin 10 (IL-10)
cagccgggaagacaataact

SEQ ID NO: 19 - DNA, 20 bp, Artificial Sequence
  Forward primer (Human Tumor Necrosis Factor alpha (TNF alpha, TNF, cachexin, or cachectin)
gaggccatttgggaacttct

SEQ ID NO: 20 - DNA, 21 bp, Artificial Sequence
  Reverse primer (Human Tumor Necrosis Factor alpha (TNF alpha, TNF, cachexin, or cachectin)
tgcctatgtctcagcctcttc

SEQ ID NO: 21 - DNA, 21 bp, Artificial Sequence
  Forward primer  (Human Interleukin-1 beta )
tcttctttgggtattgcttgg

SEQ ID NO: 22 - DNA, 21 bp, Artificial Sequence
  Reverse primer  (Human Interleukin-1 beta)
tgtaatgaaagacggcacacc

SEQ ID NO: 23 - DNA, 22 bp, Artificial Sequence
  Forward primer  (Human Hypoxanthine-Guanine Phosphoribosyltransferase 1 (HGPRT-1)
ctggttcatcatcgctaatcac

SEQ ID NO: 24 - DNA, 18 bp, Artificial Sequence
  Reverse primer (Human Hypoxanthine-Guanine Phosphoribosyltransferase 1 (HGPRT-1)
ggagcggtagcacctcct

SEQ ID NO: 25 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 26 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 27 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 28 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 29 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 30 - DNA, 5170 bp, Homo sapiens
gtggccggcggccggagccgactcggagcgcgcggcgccggccgggaggagccggagagcggccgggccgggcggtgggggcgccggcctgccccgcgcgccccagggagcggcaggaatgtgacaatcgcgcgcccgcgcaccgaagcactcctcgctcggctcctagggctctcgcccctctgagctgagccgggttccgcccggggctgggatcccatcaccctccacggccgtccgtccaggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaaaaaaaaaaaaaaaaa

SEQ ID NO: 31 - DNA, 4990 bp, Homo sapiens
attggcagctcttcacttgtatcttttcatatcaaaaatgggaggtgacacccagtttaaggaaaattccaaggcatttgtctcgactaatgtgaaagatgattacagtggccagaggactgccaaggctccttctcaagctgcttgagtcaatgagggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaaaaaaaaaaaaaaaaa

SEQ ID NO: 32 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 33 - protein, 296 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQ

SEQ ID NO: 34 - DNA, 1484 bp, Homo sapiens
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 35 - DNA, 1204 bp, Homo sapiens
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccagtaagtgggccagggtcttctgttgagaactctgtgggtttcgctcttccttttggagacagttatcactatgacccacataccacat

SEQ ID NO: 36 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 37 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 38 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 39 - protein, 143 aa, Homo sapiens
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 40 - DNA, 1794 bp, Homo sapiens
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 41 - DNA, 1865 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagctgacttgtatgaagaaggaggtggaggaggaggagaaggtgaagacaatgctgactcaaaggagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 42 - DNA, 1802 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 43 - DNA, 1973 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacaagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 44 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 45 - DNA, 477 bp, Homo sapiens
atggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 46 - protein, 270 aa, Mus musculus
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 47 - DNA, 1974 bp, Mus musculus
aagtctccagggcagagagggagtcaactcattggcgcttgagtcggcaaagaaatcaagatggccaaagttcctgacttgtttgaagacctaaagaactgttacagtgaaaacgaagactacagttctgccattgaccatctctctctgaatcagaaatccttctatgatgcaagctatggctcacttcatgagacttgcacagatcagtttgtatctctgagaacctctgaaacgtcaaagatgtccaacttcaccttcaaggagagccgggtgacagtatcagcaacgtcaagcaacgggaagattctgaagaagagacggctgagtttcagtgagaccttcactgaagatgacctgcagtccataacccatgatctggaagagaccatccaacccagatcagcaccttacacctaccagagtgatttgagatacaaactgatgaagctcgtcaggcagaagtttgtcatgaatgattccctcaaccaaactatatatcaggatgtggacaaacactatctcagcaccacttggttaaatgacctgcaacaggaagtaaaatttgacatgtatgcctactcgtcgggaggagacgactctaaatatcctgttactctaaaaatctcagattcacaactgttcgtgagcgctcaaggagaagaccagcccgtgttgctgaaggagttgccagaaacaccaaaactcatcacaggtagtgagaccgacctcattttcttctggaaaagtatcaactctaagaactacttcacatcagctgcttatccagagctgtttattgccaccaaagaacaaagtcgggtgcacctggcacggggactgccctctatgacagacttccagatatcataaaagcagccttatttcgggagtctattcacttgggaagtgctgacagtctgtatgtaccatgtacaggaaccttcctcaccctgagtcacttgcacagcatgtgctgagtctctgtaattctaaatgaatgtttaccctctttgtaagagaagagcaaaccctagtggagccaccccgacatatgatactatctgttattttaaagagtaccctatagtttgctcagtactaatcattttaattactattctgcatggcattcttaggaggatcaaaaagactctacacatattacagatgggttaacaaagggataaaacaactgaaaagcacactcaatgcatttggaatataaattcacagaccaatctcactgtgcaccttcggcttcaaaatgccagttgagtaggataaaggtataagaacttaatgctgtcattttcaaaaggaaggggacaatagctacatctttcctacctcagtgggttttactccagtgagatcatttggatgaaatcctcctgtaacagacctcaagaaggagacagactgttgaatgttatttttaagttattttatctatgtatttataaatatatttatgataattatattatttatggaacatccttaaatcctctgagcttgacggcaccctcgcagcagggttttctaggtggtcagttagatgtagtctcctctagagctccatgctacagacttttacactttttccacagccacgaagctctccgtacattcctgcacttgggagccctttcatcatgatcttaatctgtgctgtttactttgtgcatctaaaatgataattgagtcagtctttctccctcccgtccttaaagctgtctgggtattcttacatcattcagtctcacctgtaactaacaccaaccatctaaagatggaaagagcttaactgtgacaaccacatcactgatacctgaagtttcttttctagaatgtaatcagtgtttcccctggattccaatttttttttcaaaccacagtgtcatgtaactatcaacaataacaatcaactcattattattaatcataattaaataaaacaggtttgagctg

SEQ ID NO: 48 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 49 - DNA, 1328 bp, Mus musculus
cgaggcctaataggctcatctgggatcctctccagccaagcttccttgtgcaagtgtctgaagcagctatggcaactgttcctgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaagctgtggcagctacctgtgtctttcccgtggaccttccaggatgaggacatgagcaccttcttttccttcatctttgaagaagagcccatcctctgtgactcatgggatgatgatgataacctgctggtgtgtgacgttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaaagtatgggctggactgtttctaatgccttccccagggcatgttaaggagctcccttttcgtgaatgagcagacagctcaatctccaggggactccttagtcctcggccaagacaggtcgctcagggtcacaagaaaccatggcacattctgttcaaagagagcctgtgttttcctccttgcctctgatgggcaaccacttacctatttatttatgtatttattgattggttgatctatttaagttgattcaaggggacattaggcagcactctctagaacagaacctagctgtcaacgtgtgggggatgaattggtcatagcccgcactgaggtctttcattgaagctgagaataaataggttcctataatatggatgagactttttatgaatgaagcaccagcacattgctttgatgagtatgaaataaatttcattaaaacaaacaaa

SEQ ID NO: 50 - protein, 576 aa, Mus musculus
MENMKVLLGLICLMVPLLSLEIDVCTEYPNQIVLFLSVNEIDIRKCPLTPNKMHGDTIIWYKNDSKTPISADRDSRIHQQNEHLWFVPAKVEDSGYYYCIVRNSTYCLKTKVTVTVLENDPGLCYSTQATFPQRLHIAGDGSLVCPYVSYFKDENNELPEVQWYKNCKPLLLDNVSFFGVKDKLLVRNVAEEHRGDYICRMSYTFRGKQYPVTRVIQFITIDENKRDRPVILSPRNETIEADPGSMIQLICNVTGQFSDLVYWKWNGSEIEWNDPFLAEDYQFVEHPSTKRKYTLITTLNISEVKSQFYRYPFICVVKNTNIFESAHVQLIYPVPDFKNYLIGGFIILTATIVCCVCIYKVFKVDIVLWYRDSCSGFLPSKASDGKTYDAYILYPKTLGEGSFSDLDTFVFKLLPEVLEGQFGYKLFIYGRDDYVGEDTIEVTNENVKKSRRLIIILVRDMGGFSWLGQSSEEQIAIYNALIQEGIKIVLLELEKIQDYEKMPDSIQFIKQKHGVICWSGDFQERPQSAKTRFWKNLRYQMPAQRRSPLSKHRLLTLDPVRDTKEKLPAATHLPLG

SEQ ID NO: 51 - DNA, 4739 bp, Mus musculus
tgggtcgtctgactagaagtgagctgtctgtcattcttgtgcacgccagcccagtaatcatttggaggcaaagcaaactgtaagtaatgctgtcctgggctgacttgaggaggcagttttcgttttaacagccagtgtttatttgctcagcaaacgttgtctcggggagaaatgtcgctggatgtcatcagagttcccagtgccccgaaccgtgaacaacacaaatggagaatatgaaagtgctactggggctcatttgtctcatggtgcctctgctgtcgctggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacctcatcgggggctttatcatcctcacggctacaattgtatgctgtgtgtgcatctataaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggctagcatggcaaaagtgggcaggccaagaacttcggaatatctcccatcataagaggctgcagctgggctgtgcctcccagtaaaacagtcacgaaccaaacctgtgcagtcccttgttccagatcacctggaactggattgggaagagaacaggacttggtggccaggaccgctcagagagccatggttgctcagggatgctgctccgggatgcttgactaacagtcgaggcagtgaactgggtgtagaaagcgtcaggaaatggccacatgtgtggatggtttaattagattctgtggagtctcacagtgggattgtggttgtctgaggacactttggggggtcgctgtccaagaagtggctccccaaagtataagtgcgggtgaggtttactgataccccacagtggaaggcatttcttccagatattttccagataccttggccgccaaacttcgttttcaaacactgtgttttattgtgtagaccttgaaaaccacggtttcagtgaagaaggattctgtagggttcactcttctgcagtcactttagctttctgcagaagtctagattttaaattaactgtagaaggaagtcagacacccacaggtttcttctaatcaagcttccaccctgcaatggctgtcgtgccatcttccttctgtgttttactcggctcattgcttctctggagacaaatgcaatgcttcctacccccgtcctgtcacaggcagtctgaggactcaccttcacacgtgagacttcgctcttaccccagcccccaccgctggaggggtttcaatcccccaccctggggctcttctgcgcagaagctgaagtctacgctcctcccaatctttggtcccagtgccgctgttcgccttcctgctggttgattgcagaaccacctctgagacctctgtacctgctcttggaatgtctctgtctttgccctgccgcccacttcccgtgcctatgatttgctctagcatttctgccacctgacaaggagaggagcttgctaaagtttaaagtttatttcaacgctaagatcccaaccttccgatctctgcctactctcagacttggccagccctcttgcctgtcatcccaaccctgctgcttctctgtaaaaggttgtttctttactagcagaaatcagtctgcagggaggggtagtgcgtgtatctcactgttttccgtattgggaacagatggtatagatttgccaaggcctttattttaaagtgttcttggaaaatcgtatttaaatttgcaattattctaatttgatatagagagaaactgacctatttaacaaatggactctcttaattctactggacccaggccttgaactttctggtttctgtctctgtgctctgactttttgatttcaggtctagccctgtgcacttacctcagtactcagtacttactcagtactaactcagtactcgtgggaggacacccgacccgggtgttctttgagaagcagctgagtgaggcatgttttgacaggaggaacccagttcttttcactgttggtatttatagcacctaccttcagatttttcagtatgatgtttctattttccaatactggttccctaacggtcaacatttcttctcccaactaaaaatgtaagaagaaaaaaaaatttttttctttttctctttttttttttttttgtggtacagaagttttttgtttggttatttgtttgtttgattttaactagatgactgtcttcagaataatcctcacagttctggaagattgaaaaaccaagttcacatgacaagaacaaacccggttttaagggttgaatgtagggggataccattctgtcttttagactttcaacatctgctatggtggagcacaaatgttatttaaaagcatcttctcacctacatagaagtaaacaaaacacacaatttgggattttgactcttcagttttgacctcacttctcctggatccaacctgacaggagatgactggattgctggggtagagaaggaaaggctgtccccgacagcagccagaagtctgtgggagtgaggtcattcctgacccgtgatgctttcaaggcttggtttttctgataattatttttgcctgtcatcccaaaaacgtattgacatgcgtgtatattacctgtgagtgagagttcttagaaacttggccagagaggataaaggggagagcagtgacagagaaccgccgatcttgtgacaccggagtctgaggtggaacagagccagtgtcaggaaagcccctcagcaagaaagaagagtcattgctggttcccaggggcagtcacagaggatgaaataccacatcaaggcagtgggaacttcctgcgacttctcctggtcctccccagaagtatctggtgtcagctgaagaccatgtgtcccccaatacttagatttcaccgtactttctgatggtgtttttaaaaggccaagtgttgcaaaagtttgcacgtgcaaccgtatattaatcgccagtgtttaaaactggtttatcagaatttatatttttgtctctcctgccttttgaattttacttatgacagagaaagtattgagccatgactttttaaaaataatttatatttatactggtcaataatgaaggtttttttttattattaaagaacttgattttataattctgcagcagatgccaaatgttatctatcacaaaccacaaaatttatgtacagtgcatattattgatcaataaactaaacttgttttccaataaattgttaa

SEQ ID NO: 52 - DNA, 4808 bp, Mus musculus
agtctccgcccgctgcacgcccccgcgggcggctcagagtgggagcagcgtgctgctcagaaggtcttagctggtgcggggttccaggagccgcctgggagcgcggcgcccacctagaagggcgggagcccagctggtgcccgcctgccccagagcggaaggaatgtgacccccgagcgagcgcggagcagcccttgagcggctcccagggctccggttgctcccggcctgaacgggtttagttcggagctgctggattgttgaacatcgccactggggcacccgggcacgcccaggagaatatgaaagtgctactggggctcatttgtctcatggtgcctctgctgtcgctggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacctcatcgggggctttatcatcctcacggctacaattgtatgctgtgtgtgcatctataaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggctagcatggcaaaagtgggcaggccaagaacttcggaatatctcccatcataagaggctgcagctgggctgtgcctcccagtaaaacagtcacgaaccaaacctgtgcagtcccttgttccagatcacctggaactggattgggaagagaacaggacttggtggccaggaccgctcagagagccatggttgctcagggatgctgctccgggatgcttgactaacagtcgaggcagtgaactgggtgtagaaagcgtcaggaaatggccacatgtgtggatggtttaattagattctgtggagtctcacagtgggattgtggttgtctgaggacactttggggggtcgctgtccaagaagtggctccccaaagtataagtgcgggtgaggtttactgataccccacagtggaaggcatttcttccagatattttccagataccttggccgccaaacttcgttttcaaacactgtgttttattgtgtagaccttgaaaaccacggtttcagtgaagaaggattctgtagggttcactcttctgcagtcactttagctttctgcagaagtctagattttaaattaactgtagaaggaagtcagacacccacaggtttcttctaatcaagcttccaccctgcaatggctgtcgtgccatcttccttctgtgttttactcggctcattgcttctctggagacaaatgcaatgcttcctacccccgtcctgtcacaggcagtctgaggactcaccttcacacgtgagacttcgctcttaccccagcccccaccgctggaggggtttcaatcccccaccctggggctcttctgcgcagaagctgaagtctacgctcctcccaatctttggtcccagtgccgctgttcgccttcctgctggttgattgcagaaccacctctgagacctctgtacctgctcttggaatgtctctgtctttgccctgccgcccacttcccgtgcctatgatttgctctagcatttctgccacctgacaaggagaggagcttgctaaagtttaaagtttatttcaacgctaagatcccaaccttccgatctctgcctactctcagacttggccagccctcttgcctgtcatcccaaccctgctgcttctctgtaaaaggttgtttctttactagcagaaatcagtctgcagggaggggtagtgcgtgtatctcactgttttccgtattgggaacagatggtatagatttgccaaggcctttattttaaagtgttcttggaaaatcgtatttaaatttgcaattattctaatttgatatagagagaaactgacctatttaacaaatggactctcttaattctactggacccaggccttgaactttctggtttctgtctctgtgctctgactttttgatttcaggtctagccctgtgcacttacctcagtactcagtacttactcagtactaactcagtactcgtgggaggacacccgacccgggtgttctttgagaagcagctgagtgaggcatgttttgacaggaggaacccagttcttttcactgttggtatttatagcacctaccttcagatttttcagtatgatgtttctattttccaatactggttccctaacggtcaacatttcttctcccaactaaaaatgtaagaagaaaaaaaaatttttttctttttctctttttttttttttttgtggtacagaagttttttgtttggttatttgtttgtttgattttaactagatgactgtcttcagaataatcctcacagttctggaagattgaaaaaccaagttcacatgacaagaacaaacccggttttaagggttgaatgtagggggataccattctgtcttttagactttcaacatctgctatggtggagcacaaatgttatttaaaagcatcttctcacctacatagaagtaaacaaaacacacaatttgggattttgactcttcagttttgacctcacttctcctggatccaacctgacaggagatgactggattgctggggtagagaaggaaaggctgtccccgacagcagccagaagtctgtgggagtgaggtcattcctgacccgtgatgctttcaaggcttggtttttctgataattatttttgcctgtcatcccaaaaacgtattgacatgcgtgtatattacctgtgagtgagagttcttagaaacttggccagagaggataaaggggagagcagtgacagagaaccgccgatcttgtgacaccggagtctgaggtggaacagagccagtgtcaggaaagcccctcagcaagaaagaagagtcattgctggttcccaggggcagtcacagaggatgaaataccacatcaaggcagtgggaacttcctgcgacttctcctggtcctccccagaagtatctggtgtcagctgaagaccatgtgtcccccaatacttagatttcaccgtactttctgatggtgtttttaaaaggccaagtgttgcaaaagtttgcacgtgcaaccgtatattaatcgccagtgtttaaaactggtttatcagaatttatatttttgtctctcctgccttttgaattttacttatgacagagaaagtattgagccatgactttttaaaaataatttatatttatactggtcaataatgaaggtttttttttattattaaagaacttgattttataattctgcagcagatgccaaatgttatctatcacaaaccacaaaatttatgtacagtgcatattattgatcaataaactaaacttgttttccaataaattgttaa

SEQ ID NO: 53 - protein, 410 aa, Mus musculus
MFILLVLVTGVSAFTTPTVVHTGKVSESPITSEKPTVHGDNCQFRGREFKSELRLEGEPVVLRCPLAPHSDISSSSHSFLTWSKLDSSQLIPRDEPRMWVKGNILWILPAVQQDSGTYICTFRNASHCEQMSVELKVFKNTEASLPHVSYLQISALSTTGLLVCPDLKEFISSNADGKIQWYKGAILLDKGNKEFLSAGDPTRLLISNTSMDDAGYYRCVMTFTYNGQEYNITRNIELRVKGTTTEPIPVIISPLETIPASLGSRLIVPCKVFLGTGTSSNTIVWWLANSTFISAAYPRGRVTEGLHHQYSENDENYVEVSLIFDPVTREDLHTDFKCVASNPRSSQSLHTTVKEVSSTFSWSIALAPLSLIILVVGAIWMRRRCKRRAGKTYGLTKLRTDNQDFPSSPN

SEQ ID NO: 54 - DNA, 1360 bp, Mus musculus
gccttccccagtcaggcaagaagcagcaaggtacaagaatacacagctccaggctccaagggtcctgtgcgctcaggaagttggtgcggacaatgttcatcttgcttgtgttagtaactggagtttctgctttcaccactccaacagtggtgcacacaggaaaggtttctgaatcccccattacatcggagaagcccacagtccatggagacaactgtcagtttcgtggcagagagttcaaatctgaattgaggctggaaggtgaacctgtggttctgaggtgccccttggcacctcactccgacatctccagcagttcccatagttttctgacctggagtaaattggactcttctcagctgatcccaagagatgagccaaggatgtgggtgaagggtaacatactctggattctgccagcagtgcagcaagactctggtacctacatttgcacattcagaaacgcatcccactgtgagcaaatgtctgtggaactcaaggtctttaagaatactgaagcatctctgcctcatgtctcctacttgcaaatctcagctctctccaccaccgggttactagtgtgccctgacctgaaagaattcatctccagcaacgctgatggaaagatacagtggtataagggcgccatactcttggataaaggcaataaggaatttctgagtgcaggagaccccacacgcctattgatatccaacacgtccatggacgatgcaggctattacagatgtgttatgacatttacctacaatggccaggaatacaacatcactaggaatattgaactccgggtcaaaggaacaaccacggaacccatccctgtgatcatttctcccctggagacaataccagcatcattggggtcaagactgatagtcccgtgcaaagtgtttctgggaactggtacatcttccaacaccattgtgtggtggttggctaacagcacgtttatctcggctgcttacccaagaggccgtgtgaccgaggggctacaccaccagtactcagagaatgatgaaaactatgtggaagtgtcgctgatttttgatccagtcacaagggaggatctgcatacagattttaaatgtgttgcctcgaatccacggagttctcagtcactccataccacagtcaaagaagtctcttccacgttctcctggagcattgcgctggcacctctgtctctgatcatcttggttgtgggggcaatatggatgcgcagacggtgtaaacgcagggctggaaagacatatggactgaccaagctacggactgacaaccaggacttcccttccagcccaaactaaataaaggaaatgaaataaaaaaaaaaaaaaaaaaa

SEQ ID NO: 55 - protein, 178 aa, Mus musculus
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 56 - protein, 159 aa, Mus musculus
MASEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 57 - protein, 178 aa, Mus musculus; identical to SEQ ID NO: 55

SEQ ID NO: 58 - protein, 162 aa, Mus musculus
MTAAQAEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 59 - DNA, 2464 bp, Mus musculus
agttccaccctgggaaggtctgtgccatagacactgcctgggtgctcctttatacacagcaagtctctctggagtgagacgttggaaggcagtggaagaccttgtgtcctgtttagctcacccatggcttcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaagcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaaactgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttactcatccctgtgactttggctttattttacaataaaacactgaaaatgtccactttgttagttgtgaacatgagcccaggcctaaggtgctgggaaacagaaagggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcgtggttcacctcctgtgttgtttgccatgtgtgctcaggatgagctgattaaagctcttctcaggggttcagttttcccatctctgcttgtaaataaaaagcttatgcagaatactga

SEQ ID NO: 60 - DNA, 2480 bp, Mus musculus
gaatgactactttctttataagcaaccaccttgagcctgaaatggcagtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaagcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaaactgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttactcatccctgtgactttggctttattttacaataaaacactgaaaatgtccactttgttagttgtgaacatgagcccaggcctaaggtgctgggaaacagaaagggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcgtggttcacctcctgtgttgtttgccatgtgtgctcaggatgagctgattaaagctcttctcaggggttcagttttcccatctctgcttgtaaataaaaagcttatgcagaatactga

SEQ ID NO: 61 - DNA, 2409 bp, Mus musculus
agtaaaggtttctctttttggaaatatgagggtttttccgcttctgacagtggaacggaatgacagcagcacaggctgaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtactgccgaggcctgtaataatcaccaactgcctgatcactctggccatcattggggcctgaggaacaacttttgcagggtgtatgtacagtagaaggagacagaagagttctgatgatagatctctgcctcagtctgttggctggcctaatccccatgatgattccagaataatcttgcaaattggatcatggcaggtgcttgttcaaagccctttcttgttgcctctgccatctgggtgaagtctagaccacttgcttggcctaggtgtcttctgctctaccacccaccctacccctgccacaaacacacactttttttgtttttgttttttccattgttctgcacttccacagtccagaccaatcaagtcacttgacaatatgccccaagtgactcccttaccctgttttataaacctgtgcctgtctatggagaaggttttaattctccttgttattcattttgggctttttgatgaaaccaccagggcatcacatatactaagcatgtgctctaccatcatgctatgcttccagctcaggggggcacttttaaggatctagaaaacagaaattaaggatctcatagttattttattaggccagccttattccatgtcggcaagaggtttcttgtggaaattatgtcctttctgagaggagctggggattagatgctcctgcatttgtgaaatggttataagcatagaaaaataggtggtaagctttccttctttccttattttgtgtgatgccttaaactgaaaagttaaaaattgatggattgtagcattcccataatctcccccttctttttttttcctttggaaatgtccaatagtctatattcctctgtcccgcccaaacaccatcttcactccaagcctaccacagatgcctgaagaagttcctcactatctgcaaatgtggctctcaggcccttcctgatgtgatgaatgaatctactaatcatttcttgaccattcattttatcacttctaaccttgaaacatgtggaagtagctatgttcctgactgtttcctctgccagacaatgaactctggagatcagggagcttcgtgtgtgtgtgtgtgtgtgtgtgtgtgtgtgtgtgtgtgtgtgcgtgcgcgcgcgcgtgcgcacgcacgtgcatgcacatgctatgtattgggtccctccaaggatgaaccctctctttggcttagaaggcactcagagaatatgtgttattcgtgctcacggaaagtttcttactcatccctgtgactttggctttattttacaataaaacactgaaaatgtccactttgttagttgtgaacatgagcccaggcctaaggtgctgggaaacagaaagggcgggagatttttctttattctatggctagaaaatagttacctcctctctgaaagtcttcttcctcatttctgggtaacagaatatcaaacaccttgcttataagttataaagtagtgttgtccaccatgaacccaccaagtaaaaacaacccaaatacctatcatggatgaataatcatgcaagtatcagatctgcactcaatgccacacaatgacaaagatagcaaatgagccacagacggctccacccaacccaatagatgaacacttggttcaaaatcactaaagctcaaatactcccaggtcaaacaccaggtaacaagttaatactcaacaaagggggaaaacaaatgttccactgaatcctgtgaccctgtgggcgtggttcacctcctgtgttgtttgccatgtgtgctcaggatgagctgattaaagctcttctcaggggttcagttttcccatctctgcttgtaaataaaaagcttatgcagaatactga

SEQ ID NO: 62 - protein, 160 aa, Mus musculus
MNKEKELRAASPSLRHVQDLSSRVWILQNNILTAVPRKEQTVPVTITLLPCQYLDTLETNRGDPTYMGVQRPMSCLFCTKDGEQPVLQLGEGNIMEMYNKKEPVKASLFYHKKSGTTSTFESAAFPGWFIAVCSKGSCPLILTQELGEIFITDFEMIVVH

SEQ ID NO: 63 - DNA, 883 bp, Mus musculus
taacctcctcttttcgtgttgtggataaaaggcttggaccttagtagagcttgtcagagaagctgagcacagtagtgggtgtagttctgtagtgtgcagacacattcctattcaatcagggtcaatctgcagattggcagctcagaaacaacatcaccataatgaataaggagaaagaactaagagcagcatcaccttcgcttagacatgttcaggatcttagtagtcgtgtgtggatcctgcagaacaatatcctcactgcagtcccaaggaaagagcaaacagttccagtcactattaccttgctcccatgccaatatctggacactcttgagacgaacaggggggatcccacgtacatgggagtgcaaaggccgatgagctgcctgttctgcacaaaggatggggagcagcctgtgctacagcttggggaagggaacataatggaaatgtacaacaaaaaggaacctgtaaaagcctctctcttctatcacaagaagagtggtacaacctctacatttgagtctgcagccttccctggttggttcatcgctgtctgctctaaagggagctgcccactcattctgacccaagaactgggggaaatcttcatcactgacttcgagatgattgtggtacattaaggtttttagacacattgctctgtggcactctctcaagatttcttggattctaacaagaagcaatcaaagacacccctaacaaaatggaagactgaaaagaaagctgagccctccctgggctgtttttccttggtggtgaatcagatgcagaacatcttaccatgttttcatccaaagcatttactgttggtttttacaaggagtgaattttttaaaataaaatcatttatctcatttgc